SEQUENCE LISTING (11 sequences)

SEQ ID NO: 1 - DNA, 1263 bp, Homo sapiens
atgcgggcatggactggttcctggcgttggattatgctcattctttttgcctgggggaccttgttgttttatataggtggtcatttggttcgagataatgaccaccctgatcactctagcagagaactctccaagattcttgcaaagcttgaacgcttaaaacaacaaaatgaagacttgaggcgaatggctgagtctctacgaataccagaaggccccattgaccaggggacggctacgggaagagtccgtgttttagaagaacagcttgttaaggccaaagaacagattgaaaattacaagaaacaagccagaaatggtctggggaaggatcatgaaatcttaaggaggaggattgaaaatggagctaaagagctctggttttttctacaaagtgaactgaagaaattaaagcatttagaaggaaatgaactccaaagacatgcagatgaaattcttttggatttaggacaccatgaaaggtctatcatgacggatctatactacctcagtcaaacagatggagcaggggattggcgtgaaaaagaggccaaagatctgacagagctggtccagcggagaataacttatctccagaatcccaaggactgcagcaaagccaggaagctggtgtgtaacatcaataagggctgtggctatggttgccaactccatcacgtggtctactgtttcatgattgcttatggcacccagcgaacactcatcttggaatctcagaattggcgctatgctactggtggatgggagactgtgtttagacctgtaagtgagacatgcacagacagatctggcctctccactggacactggtcaggtgaagtgaatgacaaaaatattcaagtggtggagctccccattgtagacagcctccatcctcggcctccttacttaccactggctgttccagaagaccttgcagatcgactcgtaagagtccatggtgatcctgcagtgtggtgggtgtcccagttcgtcaaatatttgattcgtccacaaccttggctagaaaaggaaatagaagaagccaccaagaagcttggcttcaaacatccagtcattggagtccatgtcagacgcacagacaaagtgggaacagaggcagccttccatcccatcgaagagtacatggtacatgttgaagaacattttcagcttctcgcacgcagaatgcaagtggataaaaaaagagtatatctggctaccgatgaccctgctttgttaaaggaggcaaagacaaagtaa

SEQ ID NO: 2 - DNA, 18 bp, Rattus norvegicus
atggcacccagcgaacac

SEQ ID NO: 3 - DNA, 23 bp, Rattus norvegicus
ctactggtggatgggagactgtg

SEQ ID NO: 4 - DNA, 23 bp, Rattus norvegicus
cttggcttcaaacatccagtcat

SEQ ID NO: 5 - DNA, 21 bp, Rattus norvegicus
gaacagaggcagccttccatc

SEQ ID NO: 6 - DNA, 30 bp, Rattus norvegicus
tcatcttggaatctcagaattggcgctatg

SEQ ID NO: 7 - DNA, 26 bp, Rattus norvegicus
tggagtccatgtcagacgcacagaca

SEQ ID NO: 8 - protein, 447 aa, Artificial
  Humanized monoclonal antibody heavy chain based on murine CDRs
QVQLVESGGGVVQPGRSLRLSCKASGFNIKDYYMHWVRQAPGKGLEWIGLIDPENGNTIYDPKFQGRFTISADNSKNTLFLQMDSLRPEDTAVYYCARDNSYYFDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, Artificial
  Humanized monoclonal antibody based on murine light chain CDRs
DIQMTQSPSSLSASVGDRVTITCKASQDIRKYLNWYQQKPGKAPKLLIYYATSLADGVPSRFSGSGSGTDYTFTISSLQPEDIATYYCLQHGESPYTFGQGTKLEITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 456 aa, Homo sapiens
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 215 aa, Homo sapiens
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2009/0214528 A1
(43) Pub. Date: Aug. 27, 2009

(54) HOST CELL LINES FOR PRODUCTION OF ANTIBODY CONSTANT REGION WITH ENHANCED EFFECTOR FUNCTION

(76) Inventors: Haimanti Dorai, Radnor, PA (US); Yun Seung Kyung, Radnor, PA (US); Bernard Scallon, Radnor, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/065,178

(22) PCT Filed: Aug. 31, 2006
(86) PCT No.: PCT/US06/34382

§ 371 Date: Feb. 25, 2009

Related U.S. Application Data
(60) Provisional application No. 60/713,055, filed on Aug. 31, 2005.
(60) Provisional application No. 60/712,858, filed on Aug. 31, 2005.

(51) Int. Cl. A61K 39/395; C12N 5/06; C07K 16/18; C12P 21/08

(52) U.S. Cl. 424/133.1; 435/326; 530/387.3; 435/69.6

(57) ABSTRACT

Host cell lines for biopharmaceutical production of antibodies, antibody fragments or antibody-derived fusion proteins are selected as having the capability of inducing improved cellular effector functions, e.g., Fc-medicated effector functions. The host cells are derived from the rat myeloma cell line YB2/0 and are adapted to growth in chemically-defined medium.

### CROSS-REFERENCE TO RELATED APPLICATIONS

This application is a US National Stage of International Application Number PCT/US2006/034382, with international filing date of 31 Aug. 2006, which claims priority to U.S. Provisional Application No. 60/713,055, filed 31 Aug. 2005 and 60/712,858, filed 31 Aug. 2005. The entire contents of each of the foregoing applications is incorporated herein by reference in their entireties.

### FIELD OF THE INVENTION

The present invention relates to cells, cell lines, and cell cultures useful in recombinant DNA technologies and for the production of proteins in cell culture. More specifically, the present invention is directed to clonal myeloma cell lines capable of growing in chemically defined media that provide enhanced antibody effector function.

### BACKGROUND OF THE INVENTION

Antibodies are often referred to as adaptor molecules linking humoral and cellular immune mechanisms: humoral responses being attributed mainly to mature, secreted, circulating antibodies capable of high affinity binding to a target antigen as conferred by the inherent specificity of the variable domains. Cellular responses are attributed to the consequences of cellular activation by binding of antibody-antigen (ab-ag) complexes and by downstream sequelae caused by the release of cell mediators as a result of ab-ag complex binding to effector cells. These cellular responses include neutralization of target, opsonization and sensitization (if antigen is displayed on the surface of a cell), sensitization of mast cells, and activation of complement. For cellular targets, that is cell surface antigens, these effector functions lead to what is commonly known as Antibody Directed Cellular cytotoxicity (ADCC) and Complement-mediated cytotoxicity (CDC).

It is the so-called variable regions and hypervariable domains of the antibody that are responsible for specific antigenic recognition and the so-called constant regions of the heavy chain portion of the heterodimer, the Fc portion, that interact with these Fc-receptors present on various, usually highly motile cells, capable of stimulating those cells to affect certain functions including antibody uptake and cytotoxic mechanisms or ADCC, CDC, and also affect the antibody binding to various receptors including binding to Clq protein. These receptors are known as Fc-receptors.

Among antibody isotypes (e.g., IgA, IgE, IgD, IgG, and IgM), IgGs are the most abundant with the IgG1 subclasses exhibiting the most significant degree and array of effector functions. IgG1-type antibodies are the most commonly used antibodies in cancer immunotherapy. Structurally, the IgG hinge region and CH2 domains play a major role in the antibody effector functions. The N-linked oligosaccharides present in the Fc region (formed by the dimerization of the hinge, CH2 and CH3 domains) affects the effector functions (FIG. 1). The Fc portion of all naturally occurring antibodies are further decorated at conserved positions in the heavy chain with carbohydrate chains. In the IgG isotypes, the N-linked glycosylation site is at Asn297 which lies in each CH2 domain. As the constant regions vary with isotype, each isotype possesses a distinct array of N-linked carbohydrate structures, which variably affect protein assembly, secretion or functional activity (Wright, A., and Morrison, S. L., Trends Biotech. 15:26-32 (1997)). The structure of the attached N-linked carbohydrate varies considerably, depending on the degree of processing, and can include high-mannose, multiply-branched as well as biantennary complex oligosaccharides and sialic acid (N-acetyl neuraminic acid or NANA), fucose, galactose and GlcNAc (N-acetyl glucosamine) residues as terminal sugars shown in FIG. 2. The impact on effector functions of the host cell and oligosaccharide content of the antibodies has been recognized (Lifely, M. R., et al., 1995 Glycobiology 5:813-822; Jefferis, R., et al., 1998 Immunol Rev. 163:59-76; Wright, A. and Morrison, S. L., supra; Presta L. 2003. Curr Opin Struct Biol. 13(4):519-25). Furthermore, regarding a sugar chain in an antibody, it is reported that addition or modification of fucose at the proximal N-acetylglucosamine at the reducing end in the N-glycoside-linked sugar chain of an antibody changes the ADCC activity of the antibody significantly (WO00/61739).

Additionally, recombinant therapeutic protein production using stably engineered host cells has traditionally entailed the use of culture media supplemented with chemically undefined, animal-derived components, such as serum or organ extracts. Beyond the problem of batch-to-batch variability, the need to purify product away from these contaminants and the possibility of transmission of a human pathogen is elevated when these components are used. This sensitivity has become more acute in recent years with the discovery that Bovine Spongiform Encephalopathy (BSE), a neurodegenerative disease of cattle also known as Mad Cow Disease, is indistinguishable from the Creutzfeld-Jacob (vCJD) believed to be the pathogen for the disease affecting humans (Bruce, et al. Nature 389:498-501, 1997). Thus, many regulatory agencies strongly recommend the discontinued or limited use of animal-derived materials in cell culture media. Accordingly, chemically defined (“CD”) media for the growth and maintenance of mammalian cells which is serum-free (SF) and/or animal-derived protein-free (APF) is now available. The drawback of the CD media is that most production cell lines do not adapt to growth in it or grow slowly and produce poorly. Consequently, the ideal production cell line for manufacture of a glycosylation optimized therapeutic protein will also be capable of producing recombinant proteins at large scale, commercial capacity while growing in CD media.

Thus, in the industrial production of therapeutic recombinant proteins, there is a need for a cell line capable of affecting an optimized carbohydrate pattern on the expressed and processed proteins grown in serum-free and/or protein-free media, that improves the efficacy of the protein and obviates the need for post-harvest processing, by e.g., enzymatic means, to achieve optimized glycosylation patterns (see, for example, U.S. Pat. No. 6,399,336).

### SUMMARY OF THE INVENTION

The invention relates to cells, cell lines, and cell cultures capable of growth in chemically defined, animal-protein free medium and producing optimally glycosylated immunoglobulin-derived therapeutic proteins. In a preferred embodiment, the cell line is a YB2/0 rat myeloma derived cell line adapted to grow in CD-medium.

In a preferred embodiment, the cells, cell lines, and cell cultures of the present invention produce recombinant proteins at about 10 mg/L to about 10,000 mg/L of culture medium. In another embodiment, the cells, cell lines, and cell cultures of the present invention produce recombinant proteins at a level of about 0.1 pg/cell/day to about 100 ng/cell/day.

The present invention further provides methods for producing at least one protein, e.g., an antibody or Fc-containing protein, from a cultured host cell of the invention. In a preferred embodiment, cells of the present invention that express at least one desired protein are cultured in a chemically defined medium and the proteins are isolated from the chemically defined medium or from the cells themselves.

Another embodiment of the invention comprises an antibody or Fc-containing therapeutic protein produced by a cell line of the invention. The antibody or Fc-containing therapeutic protein of the invention can include or be derived from any mammal, such as, but not limited to, a human, a mouse, a rabbit, a rat, a rodent, a primate, or any combination thereof and includes isolated human, primate, rodent, mammalian, chimeric, humanized and/or CDR-grafted antibodies, immunoglobulins, cleavage products and other specified portions and variants thereof.

In one aspect of the invention, the antibody is an anti-integrin antibody, an anti-tissue factor antibody, or other antibody capable of binding an antigen displayed in the surface of a cell within a subject whereby reducing or preventing the growth of said cells in vivo is desirable and which activity is conferred or enhanced by production of the antibody in the cell line of the invention.

### BRIEF DESCRIPTION OF THE SEVERAL VIEWS OF THE DRAWING

FIG. 1 is a schematic depicting a typical IgG subclass mammalian antibody, domains, and glycosylation points.

FIGS. 2A-2E show the dominant biantennary oligosaccharide structures associated with natural or mammalian cell derived recombinant antibodies: abbreviated sugar structures Fuc=fucose; Gal=galactose; Glc=glucose; GlcNAc=N-acetylglucosamine; Man=mannose; and NANA*=sialyl(N-acetylneuraminic acid) identified.

FIGS. 3A and 3B show a comparison of growth and viability of APF-YB2/0 (C1083B) cell lines cultured in serum free and serum containing media over multiple generations. C1083B was cultured in DMEM+5% FBS and in CD-Hyb medium supplemented with 6 mM Glutamine. Cells were passaged three times per week using seeding density of 2−3×10⁵ cells/ml: (A) growth curve and (B) viability.

FIG. 4 shows the relative growth properties of four APF-YB2/0 cell lines derived from C1083B. Clones adapted to CD-Hyb were isolated by two methods, i.e., weaning (C1083B-1 and C1083B-12) and direct selection (C1083-H18 and C1083-H21). Cells were cultivated in CD-Hyb supplemented with 6 mM Glutamine. Cells were passaged three times per week using seeding density of 2−3×10⁵ cells/ml.

FIG. 5 is a graph demonstrating the toxicity of LCA lectin to C1083B after 5 days.

FIGS. 6A and 6B show: (A) the nucleotide sequence of rat fut8 mRNA (Genbank (NM_{—}001002289) with the location of the probe and primer sets and expression of fut8 mRNA in variants of C1083B marked (Primers (underlined) and probes (italicized) designed using the ‘Primer Express’ software (Applied Biosystems)) and (B) QPCR analyses of eight lectin-resistant cell lines derived from C1083B. Each cell line was cultured in DMEM+5% FBS and 1×10⁷ cells were harvested at exponential phase. The level of fut8 mRNA in each clone was analyzed by QPCRP.

FIGS. 7A and 7B display graphs of (A) the viable cell density and (B) viability of fucose-depleted clones derived from C1083B. The cell lines were cultured in CD-Hyb media supplemented with 6 mM Glutamine.

FIGS. 8A and 8B are a schematic representation of the CNTO 860 expression vectors used for cell line generation: (A) p2401, is the heavy chain expression vector and (B) p2402 is the light chain expression vector.

FIGS. 9A and 9B are graphs showing the stability of C1261A, a cell line expressing CNTO 860, an anti-tissue factor antibody, engineered from C1083B over time. Passage eleven cells were seeded (at 2×10⁵/ml) in duplicate in CD-Hyb medium (Gibco) in shake-flask cultures. Growth and antibody titers were monitored in the absence and presence of 1× Lipid (Gibco).

FIGS. 10A and 10B are (A) a bar graph showing dose dependent antibody specific cell lysis elicited by CNTO 859 and CNTO 860 generated in mouse myeloma line C463 and rat YB2/0 host cell line C1083B. (B) a bar graph showing the ADCC differences between CNTO 860 from C463 compared to C1083B and the fut8 depleted YB2/0 cell line C1083C (A4-3).

FIGS. 11A-C show recorder tracing from MALDI-TOF-MS analysis of CNTO860 produced by various cell lines; (A) in C463A, APF adapted rat myeloma YB2/0 host cell line, (B) C1083B, and (C) fut8 deficient YB2/0 host cell line, C1083C.

FIGS. 12A-C show recorder tracing from MALDI-TOF-MS analysis of CNTO 148 produced by various cell lines; (A) in C463A, APF adapted rat myeloma YB2/0 host cell line, (B) C1083B, and (C) fut8 deficient YB2/0 host cell line, C1083C.

FIG. 13 is a graph showing the concentration-dependence and relative ADCC activity (as measured by target cell specific lysis) for several batches of the anti-TNFalpha Mab, CNTO 148 expressed in different host cells.

FIG. 14 shows recorder tracing from MALDI-TOF-MS analysis of 2C11 anti-CD3 Mab produced by YB2/0 host cell line, C1083A.

FIG. 15 is a graph showing T-cell activation as measured by splenocyte markers on splenocytes harvested from mice that had been dosed with the various antibody preparations as noted.

### DETAILED DESCRIPTION OF THE INVENTION

### Abbreviations

Abs=antibodies, polyclonal or monoclonal; APF=animal protein free; CD=chemically defined; CDR=complementarity determining region; Ig=immunoglobulin; IgG=immunoglobulin G; Mab=monoclonal; antibody; TF=tissue factor. For sugar residues: Fuc=fucosyl; Gal=galactosyl; Glc=glucosyl; GlcNAc=N-acetylglucosaminyl; Man=mannosyl; and NANA=sialyl(N-acetylneuraminyl but can also encompass 5-N-acetylneuraminic acid (NeuAc) or 5-N— glycolylneuraminic acid (NeuGc, NGNA) as “sialic acid”; Mab=monoclonal antibody; MALDI-TOF-MS=matrix assisted laser desorption ionization time of flight mass spectrometry.

### DEFINITIONS

The term “ADCC activity” stands for antibody-dependent cell-mediated cytotoxicity and means the phenomenon of antibody-mediated target cell destruction by non-sensitized effector cells. The identity of the target cell varies, but it must have bound surface immunoglobulin G having an Fc-domain or Fc-domain portion capable of Fc-receptor activation. The effector cell is a “killer” cell possessing Fc receptors. It may be, for example, a lymphocyte lacking conventional B- or T-cell markers, or a monocyte, macrophage, or polynuclear leukocyte, depending on the identity of the target cell. The reaction is complement independent. The ADCC activity of an antibody or other Fc-containing protein of the present invention is “enhanced,” if its ability to demonstrate ADCC mediated cell killing surpasses the ability of an antibody or protein of substantially similar sequence and Fc-domain produced by an alternative host cell. ADCC activity may be determined in a standard in vivo or in vitro assay of cell killing, such as the assays discussed herein. Preferably, the antibody of the invention having enhanced ADCC activity achieves the same effect (prevention or inhibition of tumor cell growth) at a lower dose and/or in a shorter time than a reference antibody produced in an alternate host cell. Preferably, the difference between the potency of an antibody within the scope of the present invention and a reference antibody is at least about 1.5-fold, more preferably at least about 2-fold, even more preferably, at least about 3-fold, most preferably, at least about 5-fold, as determined, for example, by side-by-side comparison in a selected standard chromium release ADCC assay.

“Antibody” is intended to include whole antibody molecules, antibody fragments, or fusion proteins that include a region equivalent to the Fc region of an immunoglobulin.

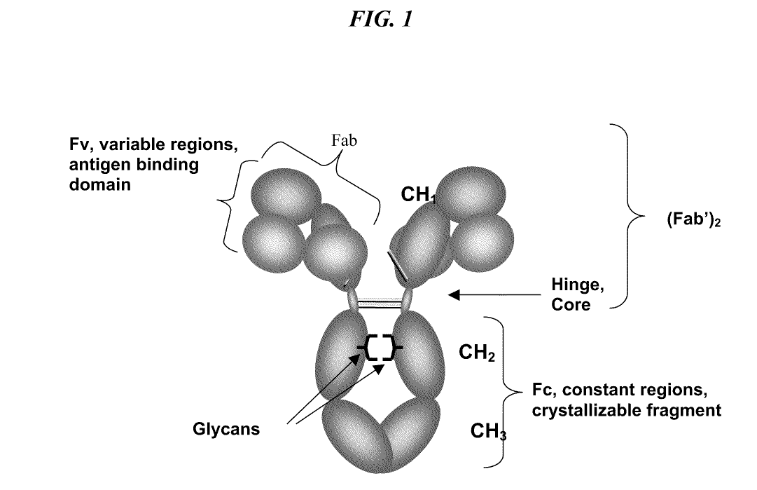

“Antibody fragments” comprise a portion of a full length antibody, generally, the antigen binding or variable domain thereof. Examples of antibody fragments include Fab, Fab′, F(ab′)2, and Fv fragments; diabodies; linear antibodies; single-chain antibody molecules; and multispecific antibodies formed from antibody fragments. Such antibody fragments may be fused to Fc-domain regions of antibodies from the same or different species or to modified Fc-domains or CH2 domains of antibodies (FIG. 1 shows the basic structure of such antibodies).

The term “cloned,” “clonally derived” or “clonal cell line” as used herein means a propagating population of genetically identical cells from a specific cell line that are derived from a single progenitor cell. For the YB2/0-derived host cells, the parental cell line is a rat myeloma cell line described in U.S. Pat. No. 4,472,500 and deposited as ATCC CRL 1662.

“Effector functions” of antibodies or antibody analogs as it is used herein are processes by which pathogens or abnormal cells, e.g., tumor cells, are destroyed and removed from the body. Innate and adaptive immune responses use most of the same effector mechanisms to eliminate pathogens including ADCC, CA (complement activation), C1q binding, and opsinization.

The terms “Fc,” “Fc-containing protein” or “Fc-containing molecule” as used herein refer to a dimeric or heterodimeric protein having at least an immunoglobulin CH2 domain. The CH2 domains can form at least a part of the dimeric region of the protein/molecule (e.g., antibody).

Fucosyl transferase or “fut8” or “fudase” refers to the gene known as fut8 and the gene product having alpha-1,6-fucosyltransferase activity.

“Fc-containing therapeutic protein” is intended to mean a dimeric or heterodimeric protein having an antigen binding domain, an Fc region, or comprising at least an immunoglobulin CH2 domain, which Fc or CH₂-comprising portion of the antibody contains an asparagine residue capable of being glycosylated.

As used herein, the term “host cell” covers any kind of cellular system which can be engineered to generate proteins, protein fragments, or peptides of interest, including antibodies and antibody fragments. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, but also cells comprised within a transgenic animal or cultured tissue.

The terms “monoclonal antibody” or “monoclonal antibody composition” or “Mab” as used herein refer to a preparation of antibody molecules of substantially single molecular composition. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clarkson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567 and Morrison et al., Proc. Nat. Acad. Sci. USA 81:6851-6855 (1984)).

“Humanized” forms of non-human (e.g., murine) antibodies are chimeric antibodies that have substantially replaced sequence portions that were derived from non-human immunoglobulin. For the most part, humanized antibodies are human immunoglobulins (recipient antibody) in which hypervariable region (which are also known as the complementarity determining regions or CDR) residues of the recipient are replaced by hypervariable region residues from a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence. The humanized antibody optionally also will comprise at least a portion of an immunoglobulin constant region (Fc), typically that of a human IgG immunoglobulin. For further details, see Jones et al., Nature 321:522-525 (1986); Reichmann et al., Nature 332:323-329 (1988); and Presta, Curr. Op. Struct. Biol. 2:593-596 (1992).

As used herein, the term “human antibody” refers to an antibody having an amino acid sequence having variable and/or constant regions derived from human germline immunoglobulin sequences. A human antibody is “derived from” a particular germline sequence if the antibody is obtained from a system using human immunoglobulin sequences, e.g., by immunizing a transgenic mouse carrying human immunoglobulin genes or by screening a human immunoglobulin gene library, and wherein the selected human antibody is at least 90%, more preferably at least 95%, even more preferably at least 96%, 97%, 98%, or 99% identical in amino acid sequence to the amino acid sequence encoded by the germline immunoglobulin gene. The human antibodies of the invention may include amino acid residues not encoded by human germline immunoglobulin sequences (e.g., mutations introduced by random or site-specific mutagenesis in vitro or by somatic mutation in vivo) and, insofar as the hypervariable sequences or complementarity determining regions (CDR) sequences are unique determinants of the antibody specificity and not coded for in the germline, these regions should be excluded from the sequence identify analysis.

The term “recombinant antibody,” as used herein, includes all antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody,” as used herein, is intended to refer to an antibody which is substantially free of other antibodies having different antigenic specificities (e.g., an isolated antibody that specifically binds to tissue factor is substantially free of antibodies that specifically bind antigens other than tissue factor). An isolated antibody that specifically binds to an epitope, isoform or variant of human tissue factor may, however, have cross-reactivity to other related antigens, e.g., from other species (e.g., tissue factor species homologs). Moreover, an isolated antibody may be substantially free of other cellular material and/or chemicals. In one embodiment of the invention, a combination of “isolated” monoclonal antibodies having different specificities are combined in a well defined composition.

The term “bispecific molecule” is intended to include any agent, e.g., a protein, peptide, or protein or peptide complex, which has two different binding specificities. For example, the molecule may bind to, or interact with, (a) a cell surface antigen and (b) an Fc receptor on the surface of an effector cell. The term “multispecific molecule” or “heterospecific molecule” is intended to include any agent, e.g., a protein, peptide, or protein or peptide complex, which has more than two different binding specificities. For example, the molecule may bind to, or interact with, (a) a cell surface antigen, (b) an Fc receptor on the surface of an effector cell, and (c) at least one other component. Accordingly, the invention includes, but is not limited to, bispecific, trispecific, tetraspecific, and other multispecific molecules which are directed to a target protein which may be a cell surface receptor or a ligand for such a receptor, and to other targets, such as Fc receptors on effector cells.

As used herein, the term “heteroantibodies” refers to two or more antibodies, antibody binding fragments (e.g., Fab), derivatives therefrom, or antigen binding regions linked together, at least two of which have different specificities. These different specificities include a binding specificity for an Fc receptor on an effector cell, and a binding specificity for an antigen or epitope on a target cell, e.g., a tumor cell.

An optimally glycosylated immunoglobulin-derived therapeutic protein comprises recombinant proteins comprising a human or human-derived CH2 region having N-linked glycosylation sites, which sites are occupied by a glycan which confers altered (relatively enhanced or diminished) ability of said thereapeutic protein to elicit cellular immune mechanisms in vivo known collectively as effector functions.

In order to produce biopharmaceutical products, a production cell line capable of efficient and reproducible expression of a recombinant polypeptide(s) is required. The cell line is stable and bankable. The cell line is capable of growth at high density, that is at concentrations greater than 500,000 (5×10⁵) cells per ml, preferably greater than one million (1×10⁶) per ml or more of culture. A variety of host cell lines can be employed for this purpose. As the understanding of the complexities of how the cellular machinery impact the final amount and composition of a biotherapeutic product, the selection of a host cell line which will impart the needed attributes to the production and the composition of the product become more evident.

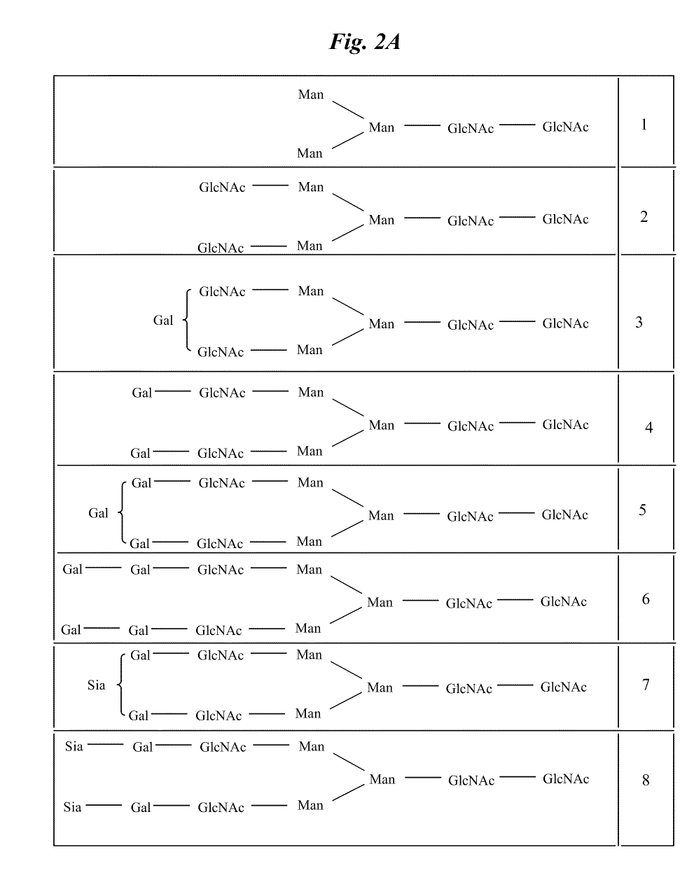

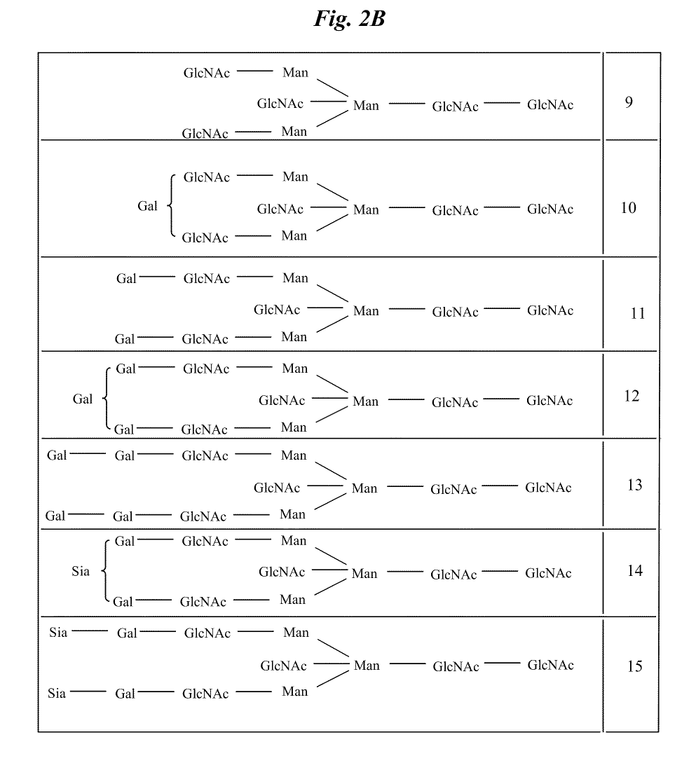

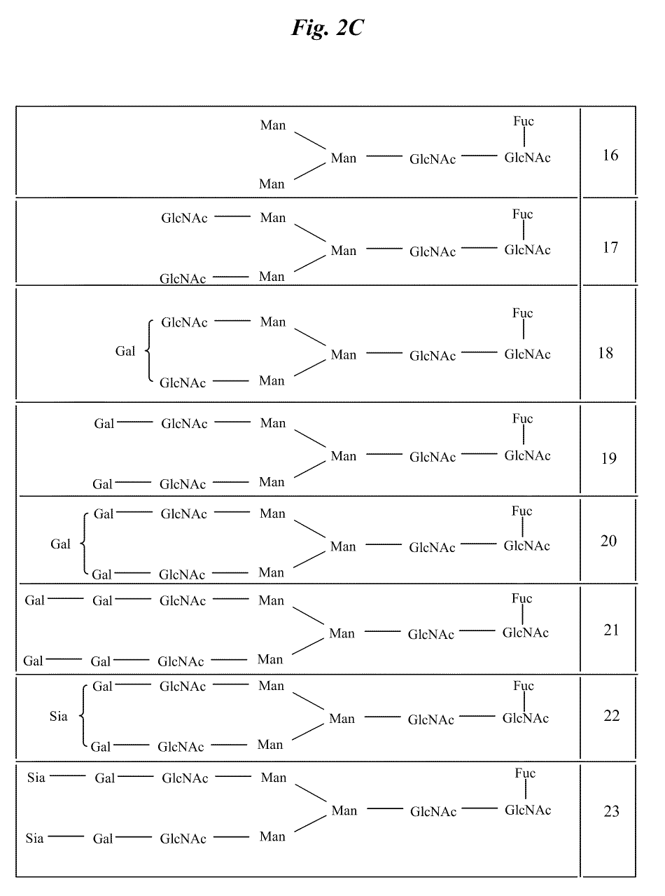

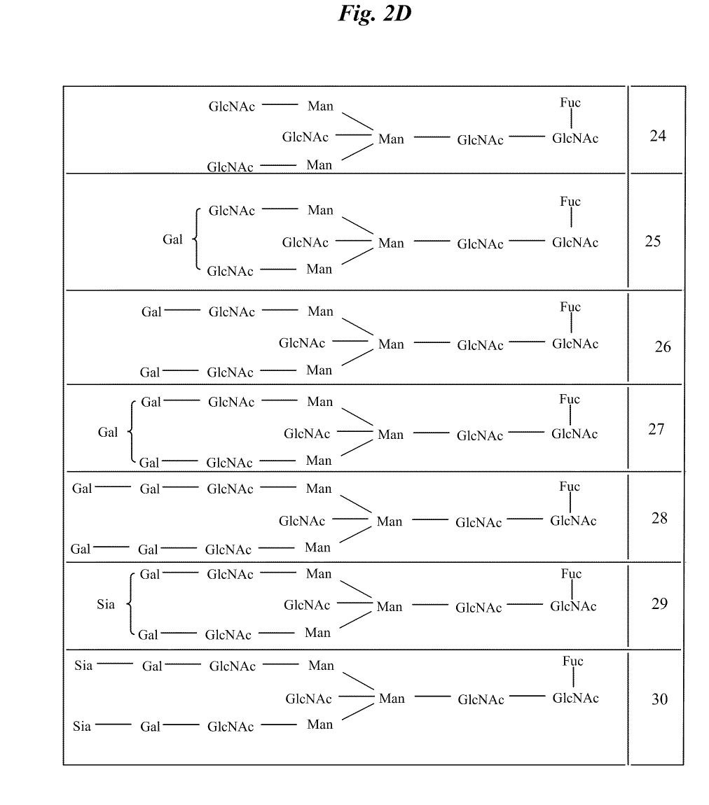

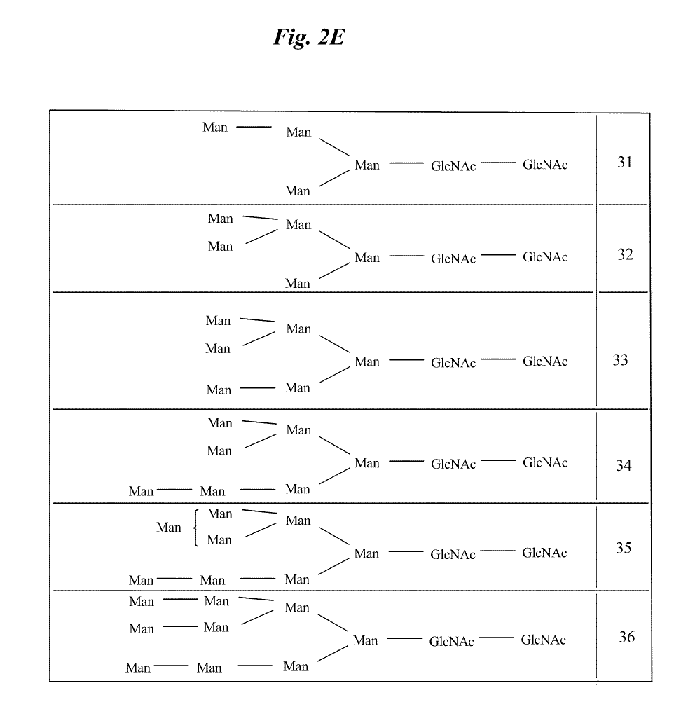

U.S. Pat. No. 4,472,500 teaches a rat myeloma cell line useful as a hybridoma fusion partner and with superior stability and production capacity. The latter cell line has been variously designated Y0, YB2 Ag0, YB2/3HL.P2.G11.16Ag.20 cell, or YB2/0 (ATCC CRL 1662) and will hereinafter be referred to as YB2/0. Lifely et al. (1995 Glycobiology 5:813-822) compared the composition of the sugar chain bound to CAMPATH-1H, a CDR-grafted human IgG1 antibody, produced by a CHO cell line, NS0 cell, or rat myeloma Y0 (YB2/0) cells. In addition, ADCC activity was assessed. It was reported that the CAMPATH-1H produced by Y0 cells showed the highest ADCC activity and had the highest content of N-acetylglucosamine (GlcNAc) at the bisecting position in the N-linked oligosaccharides (FIGS. 2A-E). This is because the glycosyl transferase that adds a bisecting GlcNAc to various types of N-linked oligosaccharides, GlcNAc-transferase III (GnT III), is not normally present in CHO cells (Stanley and Campell, 1984, J. Biol. Chem. 261:13370-13378). Other efforts to increase the ADCC capabilities of therapeutic antibodies, such as C2B8, rituximab, have focused on engineering host cell lines with optimized levels of GnT enzymes (Umana et al. U.S. Pat. No. 6,602,684). The latter inventors further discovered that overexpression of GnT III to high levels led to growth inhibition and was toxic to the cells as was overexpression of GnT V, a distinct glycosyl transferase. Thus, reduced cell viability and productivity may be a general feature of glycoprotein-modifying glycosyl transferase overexpression.

A second observation about the oligosaccharide composition of a Mab produced by the various cell hosts (Lifely supra) was that the CHO and NSO produced Mabs had predominantly fucosylated oligosaccharides (FIG. 2C-D, structures 16-30), while the YB2/0 produced Mabs had a more complex pattern which included more non-fucosylated structures (FIGS. 2A, B, & E; Structures 1-15 and 31-36).

Following this observation, it has been shown that the enzyme responsible for fucosylating the N-linked oligosaccharide structures, alpha-1,6-fucosyl transferase, the gene product offut8 and also referred to as “fudase,” was lower in YB2/0 cells than in CHO or NSO cell lines. Thus, the fut8 gene can be manipulated in host cell lines with similar effect (Shinkawa, et al. 2003 J. Biol. Chem., 278: 3466-3473; EPI 176195A1). Further, the relative contributions of galactosylation of the biantennary oligosaccharides, the presence of bisecting GlcNac, and fucosylation indicate that non-fucosylated Mabs display a greater capacity to enhance ADCC as measured in vitro and in vivo than other modifications to the N-linked biantennary oligosaccharide structures (Shields, et al. 2002. J Biol. Chem. 277:26733-40; Ninwa, et al. 2004. Cancer Res. 64:2127-2133).

### Purpose Driven Cell Line Development

Production cell line development typically involves transfection of antibody genes into host cell lines (such as the mouse myeloma Sp2/0, CD-adapted Sp2/0 (C463) and NS/0) and isolating transfectomas that express high levels of the desired antibody. In some instances, e.g., the cA2 antibody, where the therapeutic antibody acts to neutralize the biological target molecule, the antibody functions by binding and subsequently depleting the circulating TNF-α. In other instances, the antibody functions by targeting cancer cells over-expressing a particular antigen, e.g., tissue factor. While the binding of the antibody to tissue factor neutralizes tissue factor activity, the cancer cells are killed by Antibody-Dependent-Cell-Cytotoxicity (ADCC) and Complement-Dependent-Cytotoxicity (CDC) pathways activated by the recognition of bound Fc. ADCC, a lytic attack on antibody-targeted cells, is triggered upon binding of the lymphocytic receptors, FcγRs, to the constant region (Fc) of the antibodies.

### Compositions of the Invention

The present invention relates to clonal myeloma cell lines that have the ability to grow continuously in CD media. In one embodiment, the clonal myeloma cell line is a spontaneous mutant cloned from a YB2/0 cell bank in by gradually weaning the culture from FBS-supplemented CD-Hyb (CD-hybridoma, Gibco) media over six passages. In this embodiment, the clonal myeloma cell line is designated C1083B. Characterization of C1083B revealed that the cell line has a number of unique growth characteristics not associated with parental YB2/0 cells. For example, C1083B may be frozen and thawed in the absence of serum, a necessary cryopreservation agent for YB2/0 parental cell lines. In addition, unlike parental lines, C1083B can grow to high cell density in CD media. Further characterization demonstrated that C1083B grown in CD media exhibits growth parameters, including viable cell density and doubling time, that are similar or superior to those observed when cells are maintained in growth medium supplemented with serum. A second subclone of C1083A, designated C1083E, was selected by expansion of a C1083A cell culture directly into CD-Hyb medium supplemented only with 6 mM glutamine for three weeks.

In another embodiment, the clonal myeloma cell line is derived from a C1083B cells bank by selection with lectin supplemented CD medium. The lectin used in this case is Lens Culinaris Agglutinin (LCA); however, either of the two fucose-specific lectins may be used for selection. In this embodiment, the clonal myeloma cell lines are designated C1083C and C1083D. Characterization of C1083C and C1083D growth demonstrated that they were comparable to C1083B in CD-Hyb.

Therefore, C1083B cells and derivatives are capable of indefinite maintenance, growth, and proliferation in vitro. C1083B cells proliferate, can be subcultured (i.e., passaged repeatedly into new culture vessels), and cryo-preserved over time (e.g., stored in the vapor phase of liquid nitrogen with a cryo-preservative, such as 10% dimethylsulfoxide or glycerol). C1083B cells can be maintained in long-term culture as a cell line.

For the most part, cells of the invention are grown in any vessel, flask, tissue culture dish or device used for culturing cells that provides a suitably sterile environment capable of gas exchange. Typically, a foundative culture used in the invention, is one in which cells are removed from an existing parental C1083B cell stock, placed in a culture vessel in a mixture of serum containing and serum-free medium, and subsequently passaged to serum-free status as described in detail herein.

In a preferred embodiment, the cells, cell lines, and cell cultures of the present invention may produce an immunoglobulin or fragment thereof derived from a rodent or a primate. More specifically, the immunoglobulin or fragment thereof may be derived from a mouse or a human. Alternatively, the immunoglobulin or fragment thereof may be chimeric or engineered. Indeed, the present invention further contemplates cells, cell lines, and cell cultures that produce an immunoglobulin or fragment thereof which is humanized, CDR-grafted, phage displayed, transgenic mouse-produced, optimized, mutagenized, randomized or recombined.

Antibody class or isotype (IgA, IgD, IgE, IgG, or IgM) is conferred by the constant regions that are encoded by heavy chain constant region genes. Among human IgG class, there are four subclasses or subtypes: IgG1, IgG2, IgG3 and IgG4 named in order of their natural abundance in serum starting from highest to lowest. IgA antibodies are found as two subclasses, IgA1 and IgA2. As used herein, “isotype switching” also refers to a change between IgG subclasses or subtypes.

The cells, cell lines, and cell cultures of the present invention may produce an immunoglobulin or fragment thereof including, but not limited to, IgG 1, IgG2, IgG3, IgG4, IgA1, IgA2, slgA, IgD, IgE, and any structural or functional analog thereof. In a specific embodiment, the immunoglobulin expressed in the cells, cell lines, and cell cultures of the present invention is CNTO860 (cCLB8 variable domain fused to human huIgG1 derived constant domains).

The present invention further provides cells, cell lines, and cell cultures that express an immunoglobulin or fragment thereof capable of glycosylation in a CH2-domain which binds an antigen, a cytokine, an integrin, an antibody, a growth factor, a cell cycle protein, a hormone, a neurotransmitter, a receptor or fusion protein thereof, a blood protein, any fragment thereof, and any structural or functional analog of any of the foregoing. In a preferred embodiment, the immunoglobulin, fragment or derivative thereof binds an antigen on the surface of a target cell. In a particularly preferred embodiment the target cell is a tumor cell, a cell of the tumor vasculature, or an immune cell. In a specific embodiment, the immunoglobulin, fragment or derivative thereof binds to tissue factor. An example of the anti-tissue factor antibody of the invention is CNTO860 produced by the cell line designated C1261.

In yet another embodiment, the cells, cell lines, and cell cultures of the present invention may detectably express a fusion protein comprising a growth factor or hormone. Examples of the growth factors contemplated by the present invention include, but are not limited to, a human growth factor, a platelet derived growth factor, an epidermal growth factor, a fibroblast growth factor, a nerve growth factor, a human chorionic gonadotropin, an erythropoietin, a thrombopoeitin, a bone morphogenic protein, a transforming growth factor, an insulin-like growth factor, or a glucagon-like peptide, and any structural or functional analog thereof.

Isolated antibodies of the invention include those having antibody isotypes with ADCC activity, especially human IgG1, (e.g., IgG1κ and IgG1λ), and, less preferred are IgG2 and IgG3, or hybrid isotypes containing altered residues at specific residues in the Fc domains are their counterparts from other species. The antibodies can be full-length antibodies (e.g., IgG1) or can include only an antigen-binding portion and an Fc portion or domain capable of eliciting effector functions including ADCC, complement activation, and C1q binding.

Furthermore, the immunoglobulin fragment produced by the cells, cell lines, and cell cultures of the present invention may include, but is not limited to Fc or other CH2 domain containing structures and any structural or functional analog thereof. In one embodiment, the immunoglobulin fragment is a dimeric receptor domain fusion polypeptide. In a specific embodiment, the dimeric receptor domain fusion polypeptide is etanercept. Etanercept is a recombinant, soluble TNFα receptor molecule that is administered subcutaneously and binds to TNFα in the patient's serum, rendering it biologically inactive. Etanercept is a dimeric fusion protein consisting of the extracellular ligand-binding portion of the human 75 kilodalton (p75) tumor necrosis factor receptor (TNFR) linked to the Fc portion of human IgG1. The Fc component of etanercept contains the CH2 domain, the CH3 domain and hinge region, but not the CH1 domain of IgG1.

Other products amenable to manufacture using the cell lines of the invention include therapeutic or prophylactic proteins currently manufactured by other types of animal cell lines and having a CH₂ capable of being glycosylated. Particularly preferred are those therapeutic, glycosylated, CH₂-domain containing proteins which bind to target antigens on a cell surface, which cell type it is desirable to incapacitate or eliminate from the body. A number of such therapeutic antibodies are engineered to contain the human IgG1, especially the IgG1kappa, heavy chain which comprises a human CH1, CH2, and CH3 domain. Such therapeutic proteins include, but are not limited to:

Infliximab now sold as REMICADE®. Infliximab is a chimeric IgG1κ monoclonal antibody with an approximate molecular weight of 149,100 daltons. It is composed of human constant and murine variable regions. Infliximab binds specifically to human tumor necrosis factor alpha (TNF(alpha)) with an association constant of 10¹⁰ M⁻¹. Infliximab neutralizes the biological activity of TNF(alpha) by binding with high affinity to the soluble and transmembrane forms of TNF(alpha) and inhibits binding of TNF(alpha) with its receptors. Cells expressing transmembrane TNF(alpha) bound by infliximab can be lysed in vitro or in vivo.

Infliximab is indicated for the treatment of rheumatoid arthritis, Crohn's disease, and alkylosing spondylitis. Infliximab is given as doses of 3 to 5 mg/kg given as an intravenous infusion followed with additional similar doses at 2, 6, and/or 8 weeks thereafter and at intervals of every 8 weeks depending on the disease to be treated.

Daclizumab (sold as ZENAPAX®) is an immunosuppressive, humanized IgG1 monoclonal antibody produced by recombinant DNA technology that binds specifically to the alpha subunit (p55 alpha, CD25, or Tac subunit) of the human high-affinity interleukin-2 (IL-2) receptor that is expressed on the surface of activated lymphocytes. Daclizumab is a complementarity-determining regions (CDR) grafted mouse-human chimeric antibody. The human sequences were derived from the constant domains of human IgG1 and the variable framework regions of the Eu myeloma antibody. The murine sequences were derived from the CDRs of a murine anti-Tac antibody. Daclizumab is indicated for the prophylaxis of acute organ rejection in patients receiving renal transplants and is generally used as part of an immunosuppressive regimen that includes cyclosporine and corticosteroids.

Basiliximab (sold as SIMULECT®) is a chimeric (murine/human) monoclonal antibody produced by recombinant DNA technology, that functions as an immunosuppressive agent, specifically binding to and blocking the interleukin-2 receptor (alpha)-chain (IL-2R(alpha), also known as CD25 antigen) on the surface of activated T-lymphocytes. Based on the amino acid sequence, the calculated molecular weight of the protein is 144 kilodaltons. It is a glycoprotein obtained from fermentation of an established mouse myeloma cell line genetically engineered to express plasmids containing the human heavy and light chain constant region genes (IgG1) and mouse heavy and light chain variable region genes encoding the RFT5 antibody that binds selectively to the IL-2R(alpha). Basiliximab is indicated for the prophylaxis of acute organ rejection in patients receiving renal transplantation when used as part of an immunosuppressive regimen that includes cyclosporine and corticosteroids.

Adalimumab (sold as HUMIRA®) is a recombinant human IgG1 monoclonal antibody specific for human tumor necrosis factor (TNF). Adalimumab was created using phage display technology resulting in an antibody with human derived heavy and light chain variable regions and human IgG1 kappa constant regions. HUMIRA® is indicated for reducing signs and symptoms and inhibiting the progression of structural damage in adult patients with moderately to severely active rheumatoid arthritis who have had an inadequate response to one or more DMARDs. HUMIRA® can be used alone or in combination with MTX or other DMARDs.

Rituximab (sold as RITUXAN®) is a genetically engineered chimeric murine/human monoclonal antibody directed against the CD20 antigen found on the surface of normal and malignant B lymphocytes. The antibody is an IgG1 kappa immunoglobulin containing murine light- and heavy-chain variable region sequences and human constant region sequences. Rituximab has a binding affinity for the CD20 antigen of approximately 8.0 nM. Rituximab is indicated for the treatment of patients with relapsed or refractory, low-grade or follicular, CD20-positive, B-cell non-Hodgkin's lymphoma. RITUXAN® is given at 375 mg/m 2 IV infusion once weekly for 4 or 8 doses.

Trastuzumab (sold as HERCEPTIN®) is a recombinant DNA-derived humanized monoclonal antibody that selectively binds with high affinity in a cell-based assay (K_{d}=5 nM) to the extracellular domain of the human epidermal growth factor receptor 2 protein, HER2. The antibody is an IgG 1 kappa that contains human framework regions with the complementarity-determining regions of a murine antibody (4D5) that binds to HER2. HERCEPTIN is indicated as single agent therapy for the treatment of patients with metastatic breast cancer whose tumors overexpress the HER2 protein and who have received one or more chemotherapy regimens for their metastatic disease. HERCEPTIN® in combination with paclitaxel is indicated for treatment of patients with metastatic breast cancer whose tumors overexpress the HER2 protein and who have not received chemotherapy for their metastatic disease. The recommended dosage is an initial loading dose of 4 mg/kg trastuzumab administered as a 90-minute infusion and a weekly maintenance dose of 2 mg/kg trastuzumab which can be administered as a 30-minute infusion if the initial loading dose was well tolerated.

Alemtuzumab (sold as CAMPATH®) is a recombinant DNA-derived humanized monoclonal antibody (Campath-1H) that is directed against the 21-28 kD cell surface glycoprotein, CD52. Alemtuzumab binds to CD52, a non-modulating antigen that is present on the surface of essentially all B and T lymphocytes, a majority of monocytes, macrophages, and NK cells, a subpopulation of granulocytes, and tissues of the male reproductive system. The Campath-1H antibody is an IgG1 kappa with human variable framework and constant regions, and complementarity-determining regions from a murine (rat) monoclonal antibody (Campath-1G). Campath is indicated for the treatment of B-cell chronic lymphocytic leukemia (B-CLL) in patients who have been treated with alkylating agents and who have failed fludarabine therapy. Determination of the effectiveness of Campath is based on overall response rates. Campath is given initially at 3 mg administered as a 2 hour IV infusion daily; once tolerated the daily dose should be escalated to 10 mg and continued until tolerated. Once this dose level is tolerated, the maintenance dose of Campath 30 mg may be initiated and administered three times per week for up to 12 weeks. In most patients, escalation to 30 mg can be accomplished in 3-7 days.

Omalizumab (sold as XOLAIR®) is a recombinant humanized IgG1 (kappa) monoclonal antibody that selectively binds to human immunoglobulin E (IgE). Omalizumab inhibits the binding of IgE to the high-affinity IgE receptor (Fc(epsilon)RI) on the surface of mast cells and basophils. Reduction in surface-bound IgE on Fc(epsilon)RI-bearing cells limits the degree of release of mediators of the allergic response. Treatment with omalizumab also reduces the number of Fc(epsilon)RI receptors on basophils in atopic patients. Omalizumab is indicated for adults and adolescents (12 years of age and above) with moderate to severe persistent asthma who have a positive skin test or in vitro reactivity to a perennial aeroallergen and whose symptoms are inadequately controlled with inhaled corticosteroids. Omalizumab is administered SC every 2 or 4 weeks at a dose of 150 to 375 mg.

Efalizumab (RAPTIVA®) is an immunosuppressive recombinant humanized IgG1 kappa isotype monoclonal antibody that binds to human CD11a. Efalizumab binds to CD11a, the (alpha) subunit of leukocyte function antigen-1 (LFA-1), which is expressed on all leukocytes, and decreases cell surface expression of CD 11a. Efalizumab inhibits the binding of LFA-1 to intercellular adhesion molecule-1 (ICAM-1), thereby inhibiting the adhesion of leukocytes to other cell types. Interaction between LFA-1 and ICAM-1 contributes to the initiation and maintenance of multiple processes, including activation of T lymphocytes, adhesion of T lymphocytes to endothelial cells, and migration of T lymphocytes to sites of inflammation including psoriatic skin. Lymphocyte activation and trafficking to skin play a role in the pathophysiology of chronic plaque psoriasis. In psoriatic skin, ICAM-1 cell surface expression is upregulated on endothelium and keratinocytes. CD11a is also expressed on the surface of B lymphocytes, monocytes, neutrophils, natural killer cells, and other leukocytes. Therefore, the potential exists for efalizumab to affect the activation, adhesion, migration, and numbers of cells other than T lymphocytes. The recommended dose of RAPTIVA® is a single 0.7 mg/kg SC conditioning dose followed by weekly SC doses of 1 mg/kg (maximum single dose not to exceed a total of 200 mg).

In another embodiment, a cell line of the invention is stably transfected or otherwise engineered to express a non-immunoglobulin derived polypeptide.

In yet another embodiment, the cells, cell lines, and cell cultures of the present invention may detectably express a recombinant blood protein or other connective tissue protein. Such recombinant proteins include, but are not limited to, an erythropoietin, a thrombopoeitin, a tissue plasminogen activator, a fibrinogen, a hemoglobin, a transferrin, an albumin, a protein c, collagen, and any structural or functional analog thereof. In a specific embodiment, the cells, cell lines, and cell cultures of the present invention express tissue plasminogen activator.

The nucleic acids encoding the antibodies and proteins of this invention can be derived in several ways well known in the art. In one aspect, the antibodies are conveniently obtained from hybridomas prepared by immunizing a mouse with the peptides of the invention. The antibodies can thus be obtained using any of the hybridoma techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001), each entirely incorporated herein by reference.

In another convenient method of deriving the target binding portion of the antibody, typically the variable heavy and/or variable light domains of an antibody, these portions are selected from a library of such binding domains created in, e.g., a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol. Today 21(8) 371-8). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other libraries of target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc. Natl. Acad. Sci. USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based on fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

### Production Process

Once established as stably transfected, the YB2/0 cell line of the invention can be cryopreserved and retrieved to begin a production run. Typically, the cell line is banked at 1×10⁷ cells per vial in CD-Hybridoma medium supplemented with 10% DMSO. At the initiation of a production run, a vial of cells is thawed, the contents transferred to a flask containing 10 ml CD-Hybridoma media, and the flask incubated at 37° C./5% CO₂. Subsequently, the culture is expanded in a larger vessel, which in turn is transferred to a perfusion bioreactor of desired capacity (Deo et. al. 1996. Biotechol. Prog. 12:57-64).

For example, the clonal myeloma cell lines of the present invention may be manipulated to produce recombinant proteins at a level of about 0.01 mg/L to about 10,000 mg/L of culture medium. In another embodiment, the clonal myeloma cell lines of the present invention may be manipulated to produce recombinant proteins at a level of about 0.1 pg/cell/day to about 100 ng/cell/day.

Culture media or growth media useful in the present invention to support the expansion and maintenance of C1083B-E cells of the invention includes serum-free medium (SFM), protein-free media (PF), animal-derived component-free (ADCF) media, and chemically-defined (CD) formulations. CD media, as used in the present invention, comprises growth media that are devoid of any components of animal origin, including serum, serum proteins, hydrolysates, or compounds of unknown composition. All components of CD media have a known chemical structure, resulting in the elimination of—batch-to-batch variability discussed previously.

The CD media used in the present invention may include, but is not limited to, CD-Hybridoma, a CD medium produced by Invitrogen Corp., Carlsbad, Calif. (Cat. No. 11279). CD Hybridoma Medium is a chemically-defined, protein-free medium optimized for the growth of a variety of hybridomas and myelomas and the production of monoclonal antibodies in stationary or agitated suspension systems. CD Hybridoma Medium contains no proteins of animal, plant, or synthetic origin. There are also no undefined lysates or hydrolysates in the formulation. CD Hybridoma Medium is formulated without L-glutamine for increased stability.

Glutamine may be added as 40 ml of 200 mM L-glutamine or 40 ml of GlutaMAX™-I Supplement (also available from Invitrogen) per 1,000 ml of medium prior to use. A Hybridoma Medium Master file has been submitted to the FDA. CD Hybridoma Medium is not optimized for lipid-dependent or cholesterol-dependent cultures such as NSO-derived lines.

For growth profiles, CD-Hybridoma medium was supplemented with 1 g/L NaHCO₃ and L-Glutamine to final concentration of 6 mM. The present invention also contemplates the use of the chemically defined media, including “CDM medium,” described in PCT Publication No. WO 02/066603, entitled “Chemically Defined Medium For Cultured Mammalian Cells,” which is expressly incorporated by reference.

### Methods for Assessing Effector Function

The role of antibody glycosylation in the clearance, and therefore pharmacokinetics of therapeutic Fc containing proteins is unclear: binding to the neonatal Fc receptor (FcRn) thought responsible for IgG removal from circulation, appears unperturbed by lack of N-linked oligosaccharide on the Fc portion of an antibody.

The IgG Fc receptors (FcR) that link IgG antibody-mediated immune responses with cellular effector functions include the Fc-gamma receptors: FcRI (CD64), FcRII (CD32), and FcRIII (CD16). All three are found displayed on monocytes. However, the elaboration of these receptors on various target cells appears to occur differentially and in response to other factors. Therefore, measurement of the affinity of glycosylation-modified Fc containing biotherapeutics for Fc-gamma receptors is one appropriate measurement for predicting enhanced effector functions.

Human IgG1 Abs with low levels of fucose in their Fc glycans have been reported to have greater affinity for human CD 16 FcR and dramatically enhanced in vitro activity in ADCC assays using human PBMC effector cells (Shinkawa et al. J Biol Chem 278(5):3466-3473, 2003; Shields et al. J Biol Chem 277(30):26733-26740, 2002; Umana et al., Nat Biotech 17:176-180, 1999).

However, following reports that the affinity of such Abs for mouse CD16 and CD32 FcRs was no higher than that of high fucose Abs (Shields et al., 2002), there was less incentive to study low-fucose Abs in mice. Nevertheless, when the anti-tumor activity of a high fucose and a low fucose version of a chimeric human IgG1 Ab against CC chemokine receptor 4 were compared, no difference in their in vitro ADCC activity was observed (using mouse effector cells), however, the low fucose Ab showed more potent efficacy in vivo. No human effector cells were provided and the mice retain endogenous NK cells (Niwa et al. Cancer Res 64:2127-2133, 2004).

As the CD16 receptor on human NK cells has demonstrated enhanced sensitivity to fucose levels of IgG1 Abs, these data suggest that a mechanism distinct from what has been studied in human effector cells is operating in mice. One possibility is the more recently discovered mouse CD16-2 receptor (Mechetina et al. Immunogen 54:463-468, 2002). The extracellular domain of mouse CD16-2 has significantly higher sequence identity to human CD16A (65%) than does the better-known mouse CD16 receptor, suggesting that it may be more sensitive to fucose levels of IgGs that it binds than mouse CD16. Its reported expression in mouse macrophage-like J774 cells is consistent with the possibility that mouse macrophages expressing CD16-2 may be responsible for the greater anti-tumor activity by the low fucose Ab described by Niwa et al. (2004). Thus, the study of Fc-receptor binding by human IgG 1-type Fc containing proteins to murine effector cells is not predictive.

Another method of assessing effector functions is by using an in vitro ADCC assay in a quantitative manner. Thus, an in vitro assay can be designed to measure the ability of bound antibody to cause destruction of the cell displaying its cognate ligand by the correct selection of target and effector cell lines and assessing cell “kill” by either the inability of the cells to continue dividing or by release of internal contents, e.g. ⁵¹chromium release. The target cell may be a cell line which normally expresses a target ligand for the antibody, antibody fragment, or fusion protein of the invention or may be engineered to express and retain the target protein on its surface. An example of such an engineered cell line is the K2 cell, an Sp2/0 mouse myeloma cell line that stably expresses on its surface recombinant human TNF that remains as a transmembrane form due to the introduction of a deletion of amino acids 1-12 of the mature cytokine (Perez et al., Cell 63:251-258, 1990). This cell line is useful for assessing alterations in ADCC activity of anti-TNF antibodies, antibody fragments, or engineered anti-TNFalpha targeting fusion proteins having Fc-domains or Fc-domain activity.

The effector cells for the in vitro ADCC activity assay may be PBMC (peripheral blood monocytic cells) of human or other mammal source. PBMC effector cells can be freshly isolated from after collecting blood from donors by approved methods. Other monocytic or macrophage cells which may be used are those from derived from effusion fluids such as peritoneal exudates.

While having described the invention in general terms, the embodiments of the invention will be further disclosed in the following examples.

### Example 1

### Adaptation and Cloning of APF-YB2/0 Cell Line

The rat hybridoma cell line, YB2/0 (C1083A), cultured in DMEM supplemented with 5% FBS, (DMEM+5% FBS), was adapted to grow in an APF medium CD-Hyb, CD-Hybridoma (Gibco), by two different methods:

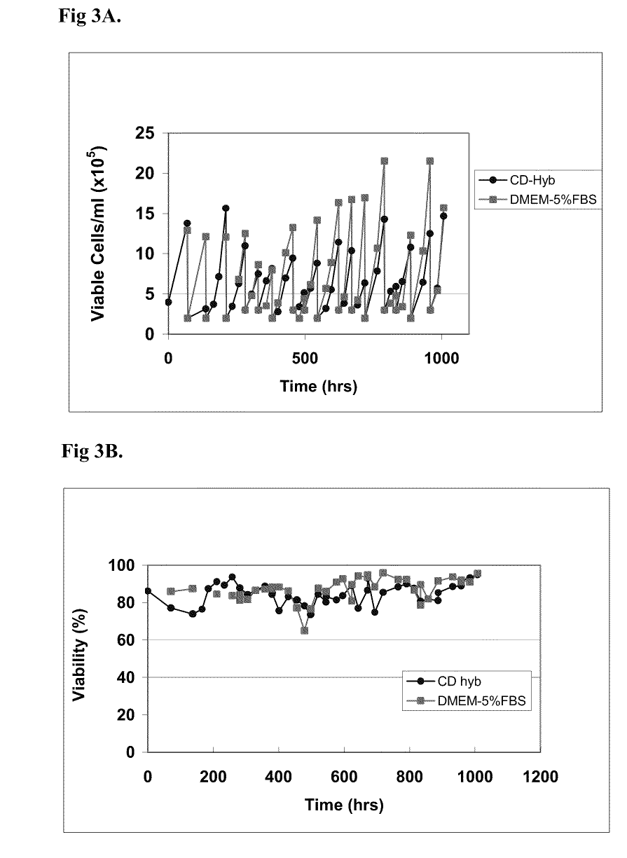

Method 1. The cells were slowly weaned from the FBS containing medium by passaging repeatedly 1:1 in CD-Hyb medium supplemented with 6 mM Glutamine. After 6 passages, the cells were capable of growth in APF medium. This cell line was designated C1083B (Table 1). Growth characteristics of C1083B in CD-Hyb and DMEM+5% FBS were comparable (FIG. 3). Individual clones from C1083B were isolated by the limiting dilution method using DMEM+5% FBS. Twenty-four clones were transferred for scale-up and eight clones from this experiment were selected for further study. The criteria for selection of these eight clones included mean doubling time (MDT), ability to reach high cell density in shake-flask cultures and stability over multiple passages.

TABLE 1

| Cell Line | Derived from | Remarks |
|---|---|---|
| C1083A | YB2/0 | ATCC CRL-1662 |
| C1083B | C1083A | Adapted to CD-Hyb, serum free media |
| C1083C | C1083B | Expresses 6-fold less fut8 mRNA |
| C1083D | C1083B | Expresses 2-fold less fut8 mRNA |
| C1083E | C1083B | Subclone of C1083B |
| C1261A | C1083B | Transfected cells secreting CNTO 860 |

Method 2. Two hundred, 500, 1000 or 5000 C1083A cells were plated per well of 96-well plates (5 plates for each category) in CD-Hyb medium supplemented with 6 mM glutamine. After three weeks of incubation, only plates with 5000 cells/well had colonies in approximately 10 wells/plate. Twenty-four clones were transferred to a 24-well plate for expansion. Four clones were picked for further study, based on the mean doubling time, ability to reach high cell density in shake-flask cultures and stability over multiple passages.

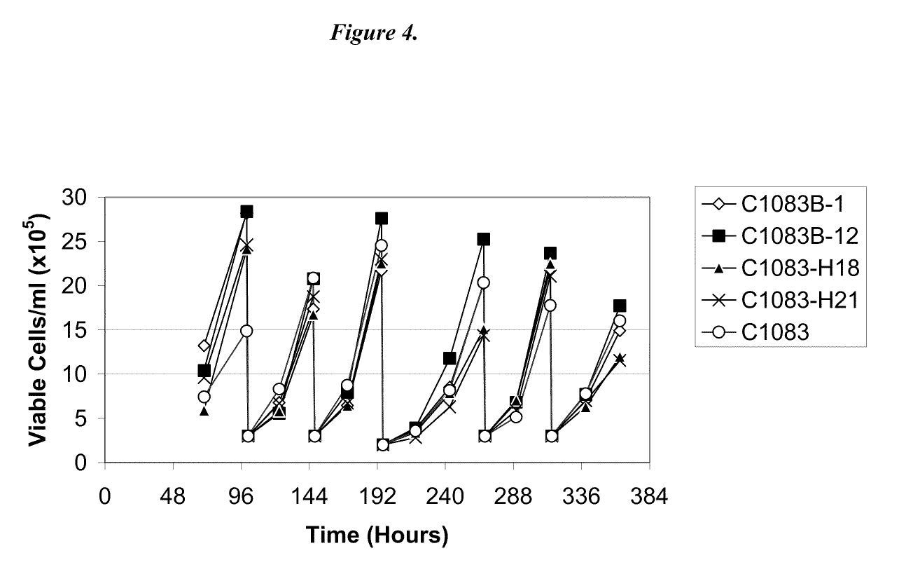

Twelve clones, eight generated by method 1 and four generated by method 2 were compared for growth characteristics in CD-Hyb medium, i.e., mean doubling time, ability to reach high cell density in shake-flask cultures and stability over multiple passages. Four clones were selected (C1083B-1, C1083B-12, C1083-H18 and C1083-H21) from this experiment for further study. All four had comparable growth characteristics. Their MDT was approximately 22 hours and they were able to reach high cell density (>2×10⁶/ml) in shake-flask cultures (FIG. 4). Three of the four cell lines, (C1083B-1, C1083B-12 and C1083-H21) were then tested to determine their transfection efficiency using the AMAXA electroporator and settings previously optimized for myeloma cell line transfections. Cell line C1083B-12 was chosen from this study as the APF-YB2/0 cell line with the desired characteristics and will serve as the alternate transfection host cell line in addition to C1083B. It was designated C1083E.

### Example 2

### Isolation of YB2/0 Clones Resistant to Fucose-Specific Lectins

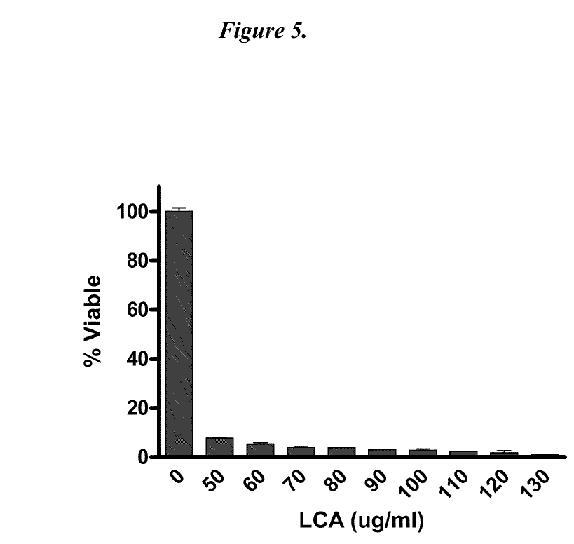

Lectins can be used to select cell lines expressing a specific type of oligosaccharide (Ripka and Stanley, 1986. Somatic Cell Mol Gen 12:51-62). Of the two fucose-specific lectins available, Lens Culinaris Agglutinin (LCA) was selected for generating a kill curve (in bar graph form) using C1083B (FIG. 5). C1083B cells (cultured in DMEM+5% FBS) were plated at 5000 cells/well in 96-well plates in the presence of various concentrations of LCA lectins. After 5 days, viability was determined by the Alamar Blue assay (Vybrant Cell Metabolic Assay Kit, Molecular Probes, Inc.).

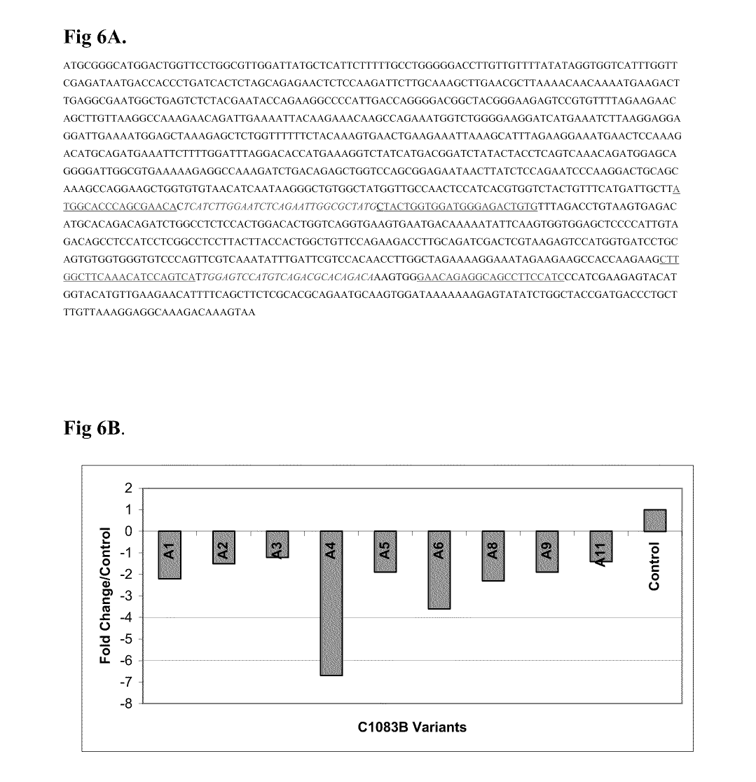

Rare natural variants of C1083B expressing reduced levels of fut8 mRNA (SEQ ID NO: 1) were selected by plating 5 cells/well in 96-well plates in the presence of 50 ug/ml LCA. After three weeks, 17 resistant clones (out of 2×10⁴ cells plated) were identified. These were scaled up and passaged multiple times in CD-Hyb medium. Eight of the 17 clones were selected based on robust growth, ability to reach high cell density in shake-flask cultures and stability of the culture over multiple passages. Total RNA was isolated from these clones. Quantitative PCR experiments using two sets of rat specific fut8 Taqman probes (underlined) and primers (italicized) (SEQ ID NOS: 2-7, FIG. 6A).

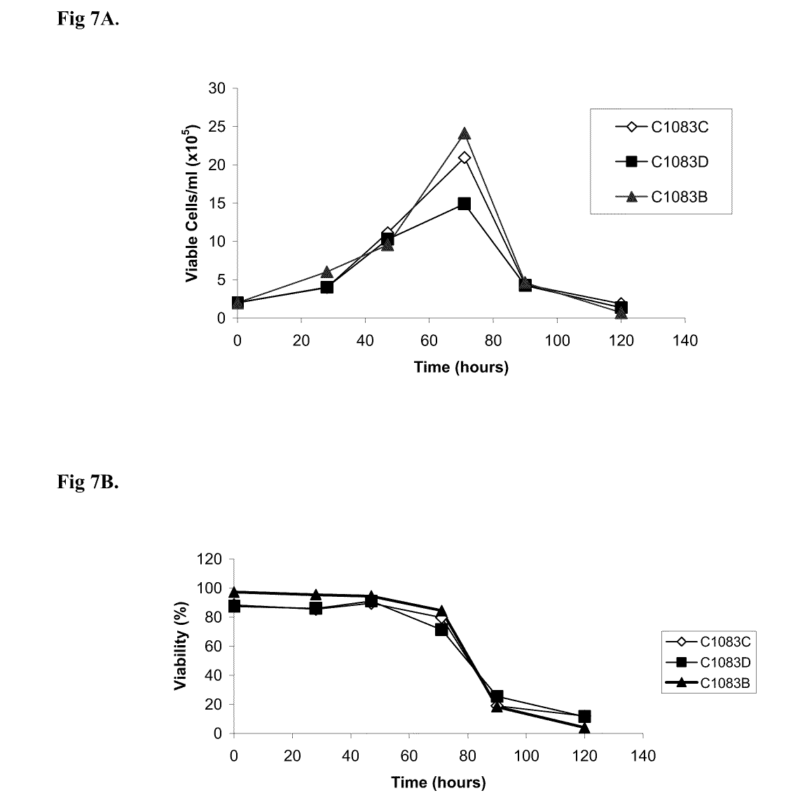

These analyses demonstrated that one clone (A4) had 6-fold less fut8 mRNA, whereas two other clones (A8 and A9) had approximately 2-fold less fut8 mRNA (FIG. 6B). Clone A4 was designated C1083C and clone A9 was designated C1083D. Data from FIGS. 7A and B demonstrate that the growth characteristics of C1083C and C1083D in CD-Hyb are comparable to those of the parental line, C1083B based on viable cells per volume of culture medium (FIG. 7A) and on total cell viability (FIG. 7B).

### Example 3

### Transfection of C1083B Cells with Anti-Tissue Factor Antibody DNA

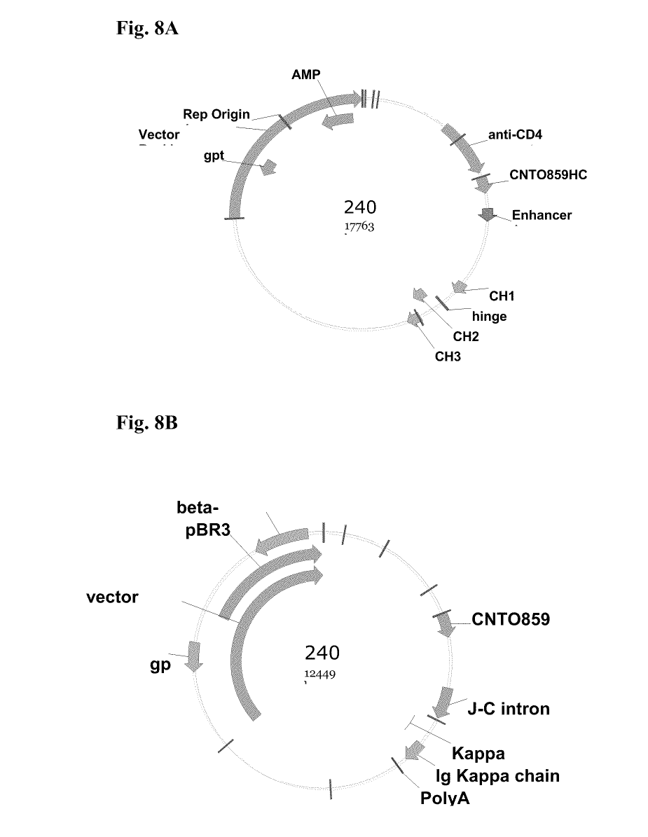

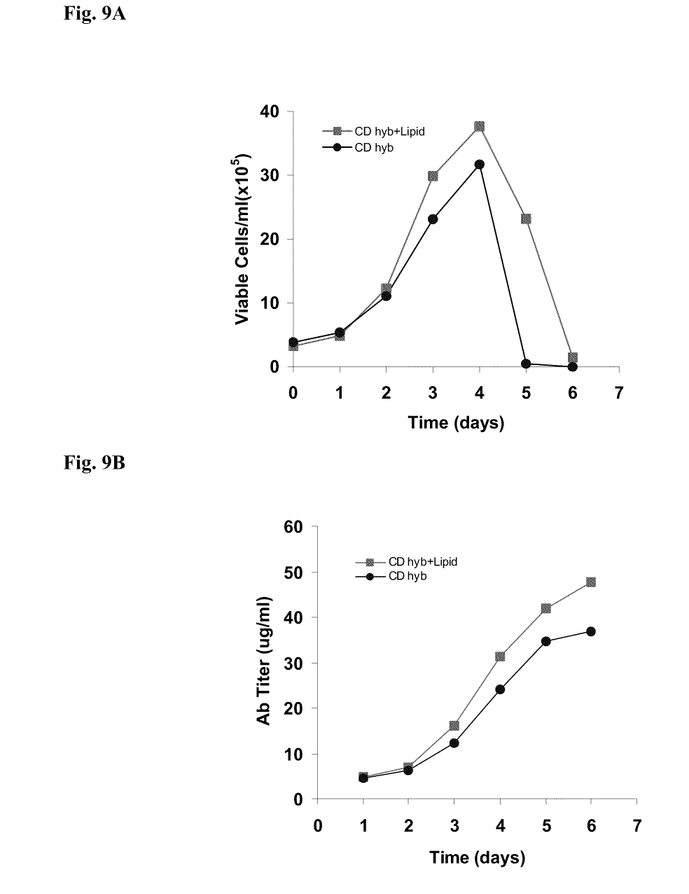

CNTO 860, an anti-human-tissue factor antibody, was selected because its efficacy in reducing or preventing tumor growth as tested in human xenografts models of cancer in mice, is dependent on ADCC activity. Expression vectors (p2401 and p2402) encoding CNTO 860 heavy and light chains, as shown in FIG. 8 are further described in WO/04110363 and U.S. patent application Ser. No. 11/010,797) were co-transfected with pSV2DHFR (Promega) and clones resistant to the selection marker MHX, (mycophenolic acid, hypoxanthine and xanthine), were analyzed for antibody expression by ELISA. One high expressing cell line, C1261A, was selected for further study. It produced 45-50 mg/L in CD-Hyb medium in shake flask cultures and demonstrated stability in expression over multiple passages (FIG. 9). Growth and antibody titers were monitored in absence and presence of 1× Lipid (Gibco).

### Example 4

### Determination of ADCC Activity of Anti-Tissue Factor Antibody Derived from C1083B

A series of in vitro ⁵¹Cr-release cytotoxicity assays were used to demonstrate the enhancement of ADCC activity of several anti-tissue factor antibodies: CNTO859, which contains a human IgG4 Fc (described in EP833911B1); CNTO860, which has the same antigen binding region as CNTO859 but has been cloned into a human IgG1 framework and thus produce an humanized antibody having the sequence of SEQ ID NO: 8 for the heavy chain and SEQ ID NO: 9 for the light chain (as described in U.S. application Ser. No. 11/010,797, filed Dec. 13, 2004); and a glycosylation variant as a result of producing the CNTO860 antibody in the YB2/0 CD-Hyb adapted cell line or fut8-deficient variants.

The tissue factor expressing human colon carcinoma cells, HCT 116, were used as target cells. Cells were maintained in McCoy's 5A medium supplemented with 10% heat-inactivated FBS and 1% LNN (M5A-10). On the day of the assay, cells were trypsinized, harvested and labeled at 10×10⁶ cells per 200 uCi of Na₂⁵¹CrO₄ (PerkinElmer Life Science, Boston, Mass.) in 1 mL M5A-10 for 2 hrs at 37° C. Labeled cells were washed twice with 50 mL PBS without calcium or magnesium (PBS⁻) and resuspended to 4×10⁵ cells/mL M5A-10.

PBMCs were isolated from healthy donors. Venous blood was collected into heparinized syringes and diluted with an equal volume of PBS⁻ into a 50 mL conical tube (20 mL: 20 mL). This blood solution was underlayed with 13 mL of Ficoll-Paque (Amersham, Uppsala, Sweden) and centrifuged at 2200 rpm for 30 minutes at room temperature (RT). The top plasma layer was aspirated and the interface (buffy layer) containing PBMCs was harvested. Effector cells were washed three times in PBS⁻ and then resuspended in M5A-10 at 5×10⁶ cells/mL. An effector-to-target ratio of 25:1 was used for all experiments.

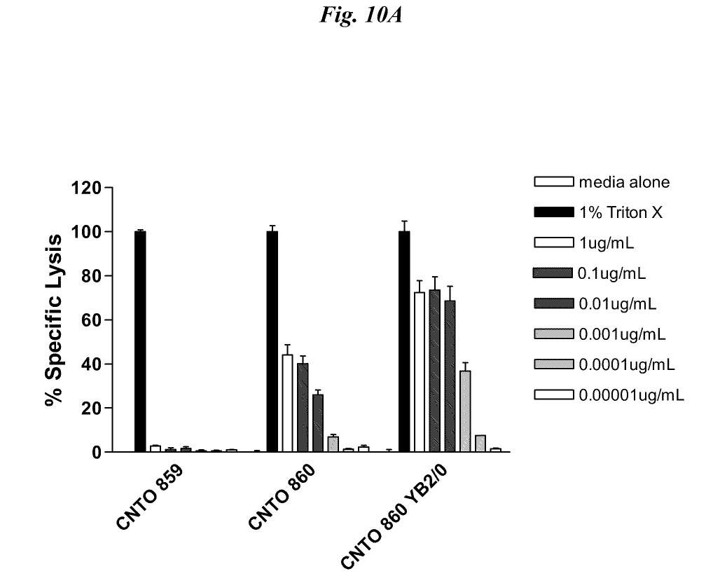

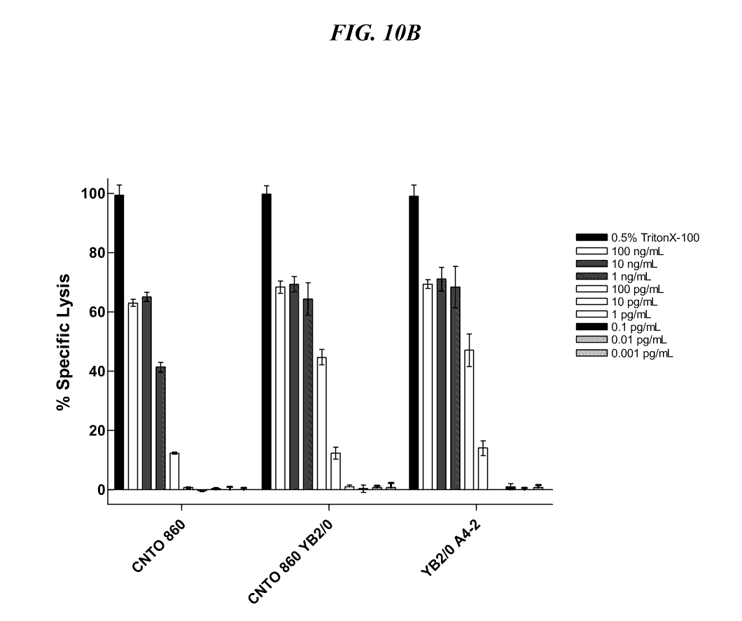

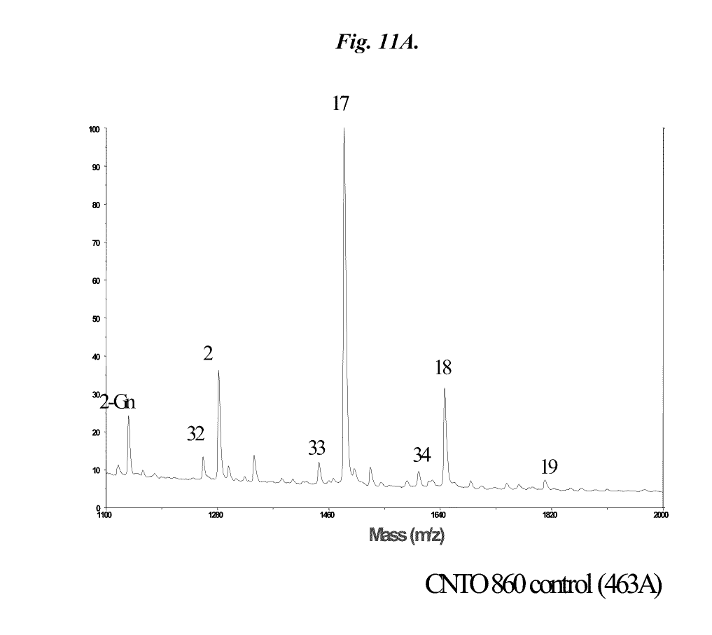

In the first experiment, the ADCC activity of the monoclonal antibodies against tissue factor, namely CNTO 859, CNTO 860 and CNTO 860 YB2/0, was characterized using PBMCs from two different donors (FIG. 10A). Specific lysis was determined after 4 hours and each bar is representative of the mean of triplicates from both donors. Spontaneous and maximal release control samples were treated with media alone in the presence of 2 μg/mL antibody but no effector cells or treated with 0.5% Triton X-100, respectively. The percentage of specific lysis in each sample was calculated based on cpm released by Triton X-100 (maximum release) corrected by the spontaneously release cpm.

CNTO 859, the IgG4 subtype, possesses minimal ADCC activity compared to CNTO 860, the IgG1 subtype produced by a mouse myeloma host cell line, C463. In contrast, CNTO 860 derived from the YB2/0 host cell line C1083B, was roughly 20-60 fold more potent than that derived from C463 (FIG. 10A) when comparing their EC₅₀ and maximal lysis values. The YB2/0 derived CNTO 860 was 40% fucosylated as compared to C463 derived CNTO 860 which was 99% fucosylated.

In a second experiment, CNTO 860 derived from 3 cell lines were compared for their relative ADCC potency, namely, C463; the animal protein-free adapted YB2/0 cell line, C1083B and the fut8 depleted YB2/0 cell line, C1083C. Specific lysis was determined after 4 hours and bars represent the mean of triplicates from a single donor.

CNTO 860 derived from the C1083B cell line was roughly 10-fold more potent than that derived from the mouse myeloma cell line, C463 (FIG. 10B).

No difference in ADCC activity was observed between CNTO 860 derived from the parental YB2/0 derived cell line, C1083B, and the fut8 depleted clone A4-2, C1083C. These results indicate that a further increase in ADCC activity by reducing the fucose level further was not measurable using the in vitro assay method.

### Example 5

### Analysis of Antibody Glycosylation

MALDI-TOF-MS analysis of CNTO 860 generated in C463 and various transfection host cell lines was performed.

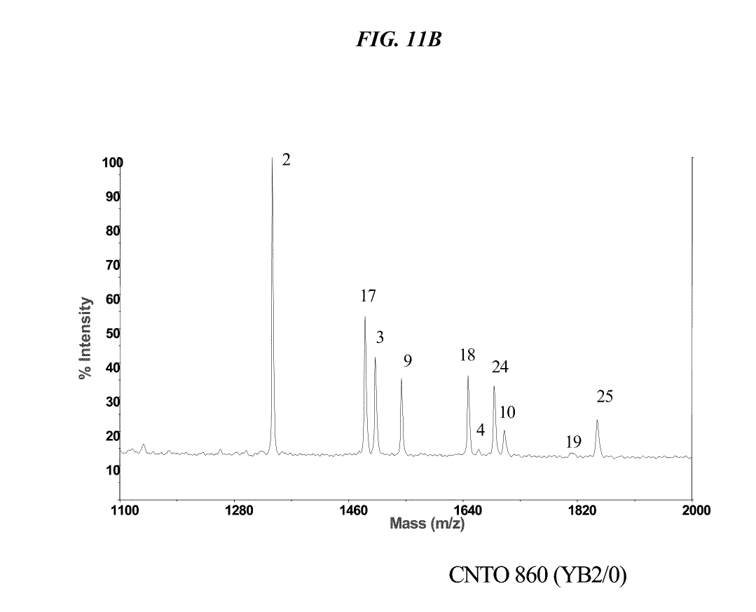

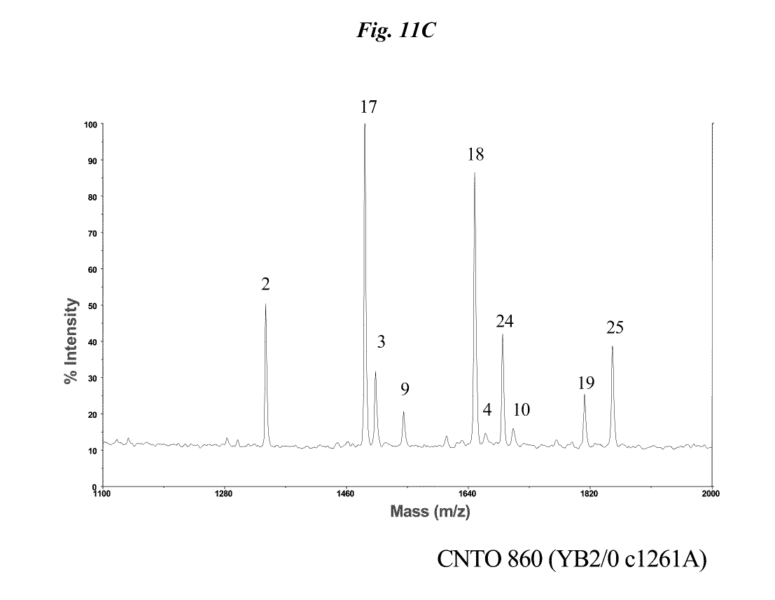

CNTO 860 generated in C463A (FIG. 11A), APF adapted rat myeloma YB2/0 host cell line, C1083B (FIG. 11B) and fut8 depleted YB2/0 host cell line, C1083C (FIG. 11C) were subjected to MALDI-TOF-MS analysis as per published protocols. (Papac et al., 1996; Papac et al., 1998; Raju et al., 2000).

Test Abs were structurally analyzed by different methods. To perform MALDI-TOF-MS analysis of intact IgG Abs, IgG samples were brought into 10 mM Tris-HCl buffer, pH 7.0 and adjusted concentration to ˜1 mg/mL buffer. About 2 μl of IgG solution was mixed with 2 μl of matrix solution (the matrix solution was prepared by dissolving 10 mg sinnapinic acid in 1.0 ml of 50% acetonitrile in water containing 0.1% trifluoroacetic acid) and 2 ml of this solution was loaded onto the target and allowed to air dry. MALDI-TOF-MS was acquired using a Voyager DE instrument from Applied BioSystems (Foster City, Calif.).

To perform MALDI-TOF-MS analysis of released Fc glycans, IgG samples (˜50 μg) were digested with PNGase F in 10 mM Tris-HCl buffer (50 μl) pH 7.0 for 4 h at 37° C. The digestion was stopped by acidifying the reaction mixture with 50% acetic acid (˜5 μl) and then passed through a cation-exchange resin column as described previously (Papac et al., 1996; Papac et al., 1998; Raju et al., 2000). These samples containing a mixture of acidic and neutral oligosaccharides were analyzed by MALDI-TOF-MS in the positive and negative ion modes, as described elsewhere (Papac et al., 1996; Papac et al., 1998; Raju et al., 2000) using a Voyager DE instrument from Applied BioSystems (Foster City, Calif.).

MALDI-TOF-MS analyses of the released glycans from antibody samples produced in different YB2/0 cells are shown in FIGS. 11A-C and the structure of the oligosaccharides are depicted in FIGS. 2A-E. The oligosaccharides are numbered in sequence based on the presence of core fucose, bisecting GlcNAc, presence or the absence of terminal sugars, such as sialic acid, galactose etc. The MALDI-TOF-MS data suggest that antibody samples produced in YB2/0 cells contain increased amounts of non-fucosylated oligosaccharides (FIG. 2A-B, structures 1-15). The amounts of non-fucosylated oligosaccharides vary from 50% to 95% for certain antibody samples. Additionally, an increase in non-fucosylated oligosaccharides containing bisecting GlcNAc was also observed in the YB2/0 derived antibody samples. Further, the antibody samples derived from YB2/0 cells contain either increased homogeneity and/or more homogeneous structures due to the presence of non-fucosylated and bisecting GlcNAc containing oligosaccharides. On the contrary the antibody samples produced in other cell types tend to contain more heterogeneous structure of oligosaccharides (FIG. 2A-E, structures 1-36) indicating the value of YB2/0 cells to produce therapeutic antibody samples with increased activity due to the presence of more defined and homogeneous oligosaccharide structures. Further, the antibody samples produced in YB2/0 cells tend to contain a lower percentage of structures with high mannose content (FIG. 2E, structures 31-36) compared to the antibody samples produced in other cell lines such as HEK or NS/0.

### Example 6

### C1083B/C Expression of Anti-TNF_{ALPHA} MAB

Examination of CNTO 860 expression levels in several myeloma host cell lines (Sp2/0, NS0 and YB2/0) revealed relatively lower levels of antibody production compared to other antibodies produced in these cell lines. Therefore, an alternate antibody was selected for expression in the YB2/0-derived host cell lines of the invention. C1083B YB2/0 cells and C1083C YB2/0 cells were transfected with heavy (the variable region of this is SEQ ID NO: 10) and light chain (the variable region of this is SEQ ID NO: 11) encoding plasmids (plasmids p1783 and p1776, respectively) encoding a human anti-TNFalpha Mab designated CNTO 148 (Golimumab), by electroporation as described (Knight et al., Mol Immunol 30:1443-1453, 1993; WO02/012502). Mycophenolic acid-resistant colonies of the transfected YB2/0-derived cells were assayed for the presence of CNTO 148 in their culture supernatants by ELISA for human IgG as described (Knight et al., 1993). The transfectants (#14 C1083B transfectant and #1 C1083C transfectant) were scaled-up in IMDM, 5% FBS, 1% glutamine, 1×MHX selection (0.5 μg/ml mycophenolic acid, 2.5 μg/ml hypoxanthine, 50 μg/ml xanthine) to a volume of 1 liter, the cultures allowed to overgrow until cell viability was <20%. Standard protein A chromotagraphy used to purify the two samples of CNTO 148. The purifications yielded 1.3 mg of CNTO 148 from the C1083B-transfected cells and 3.2 mg of CNTO 148 from the C1083C-transfected cells.

C1083B-148-14 and another clone, C1083B-148-33 were subjected to Halo subcloning. Twenty-one halos were picked from the 1st round Halo from clone 33, of which one subclone, C1083B-148-33-19, expressed ˜89 ug/mL in a shake flask. Upon expansion and a 2nd round of Halo, subclone C1083B-148-33-19-42, exhibited titers of ˜105 ug/mL in a shake flask. This clone is being adapted to APF medium.

Bioanalytical Characterizations of YB2/0-Derived CNTO 148

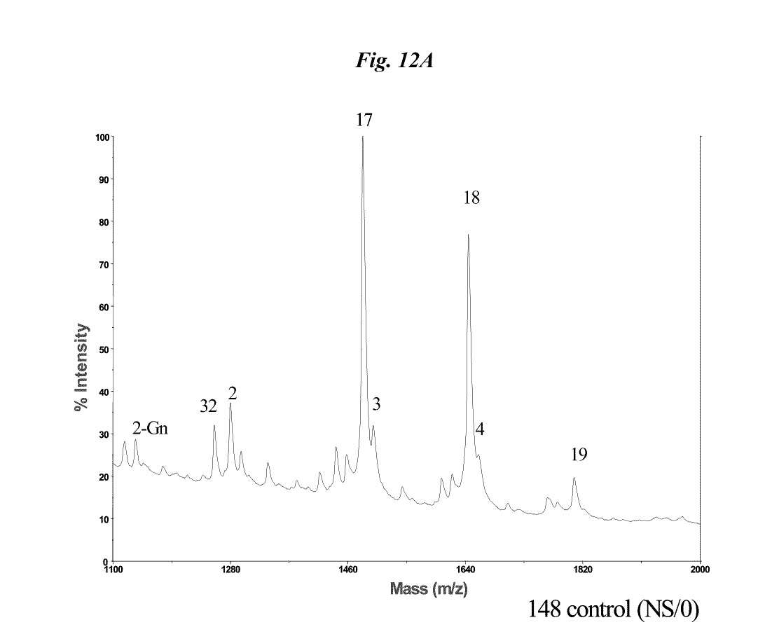

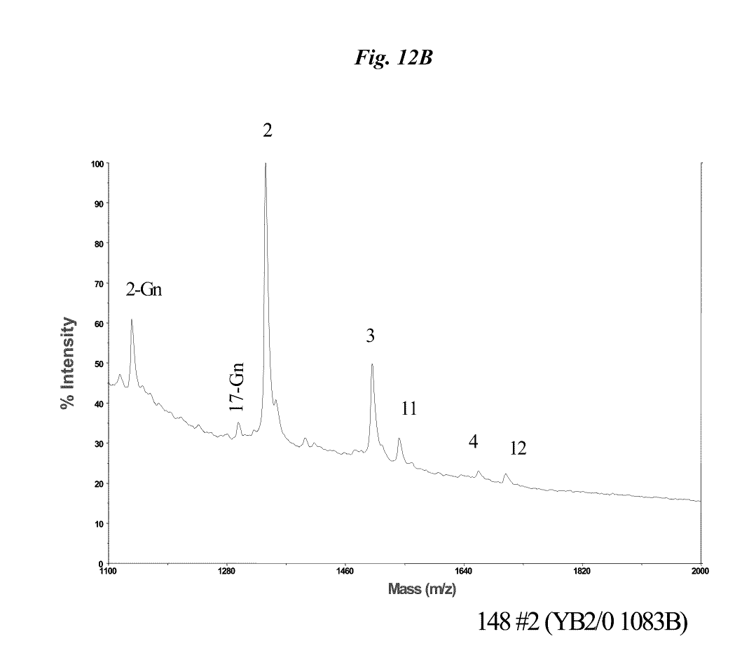

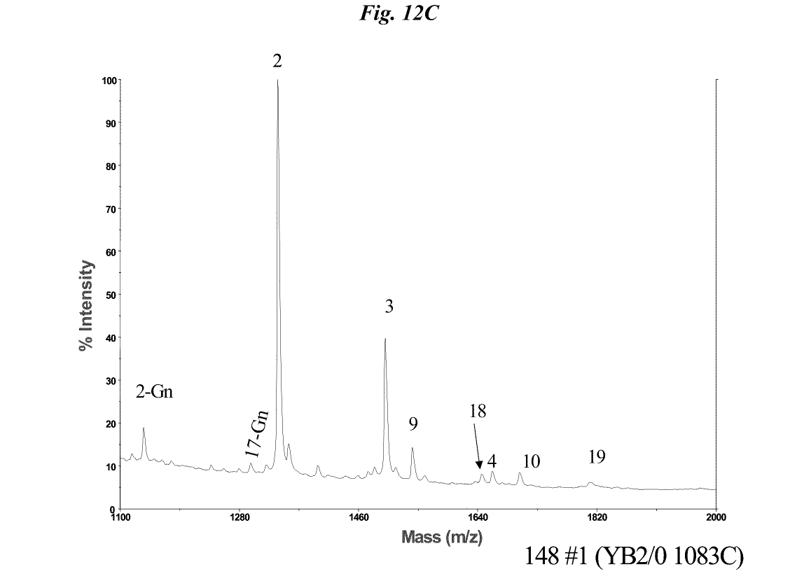

MALDI-TOF-MS analysis of the PNGase F released oligosaccharide (FIG. 12A-C) indicated that greater than 80% of the oligosaccharides from the YB2/)-derived host cells, C1083B and C1083B were not fucosylated. Unexpectedly, the fucose content of the C1083C-derived CNTO 148 was found to be no lower than that of the C1083B-derived CNTO 148 (FIGS. 12B & C). The oligosaccharides from these antibody samples also contain increased amounts of bisecting GlcNAc without fucose appear to be more homogeneous than the oligosaccharides from antibody produced in the NS/0 host cells (FIG. 12A).

In vitro ADCC assay with YB2/0-derived CNTO 148. The target cells designated K2 or C480A cells are an Sp2/0 mouse myeloma cell line that stably expresses on its surface recombinant human TNF that remains as a transmembrane form due to the introduction of a deletion of amino acids 1-12 of the mature cytokine (Perez et al., 1990 supra). K2 cells were cultured in Iscove's media containing heat inactivated FBS, 2 mM L-glutamine, 1 mM sodium pyruvate, 0.1 mM non-essential amino acids, and 1×MHX selection. The K2 cells were passaged 1:5 every 2-3 days.

On the day of the assay, K2 cells were centrifuged and washed once with PBS. Cells were adjusted to about 1×10⁶ cells/ml with the culture medium and 15 microliters of BATDA fluorescent labeling reagent (in Delfia EuTDA Cytotoxicity Reagent Kit, Perkin-Elmer Life Sciences) was added to 5 ml of cells (Blomberg et al., J Immunl Methods 193:199-206, 1996). Cells were incubated for 30 minutes at 37° C., then washed twice with PBS at 1000 rpm, for 5 min. Immediately prior to mixing with PBMC effector cells, targets cells were centrifuged and resuspended at 2×10⁵ cells/ml in Iscove's media containing 1% BSA.

PBMC effector cells were isolated from healthy donors after collecting blood into heparinized vacutainers, and diluting two-fold with PBS. Thirty (30) ml of diluted blood was layered on top of 15 ml of Ficoll-Paque (Amersham, Uppsala, Sweden) in a 50 ml conical tube and centrifuged at 1500 rpm, 30 min at RT. The interface (buffy layer) containing PBMCs was collected and washed twice with PBS and centrifuged at 1200 rpm, for 10 min, RT. Cells were resuspended in Iscove's media containing 5% heat inactivated FBS, 2 mM L-glutamine, 1 mM sodium pyruvate and 0.1 mM non-essential amino acids. PBMCs were activated for approximately 4 h at 37° C., 5% CO₂ by incubating on 100 mm tissue culture dishes that had been coated with OKT3 (10 ug/ml in PBS, Ortho Pharmaceutical) overnight at 4° C. and rinsed with PBS. PBMCs were collected, washed once with Iscove's media containing 1% BSA, counted and resuspended to approximately 1×10⁷ cells/ml.

CNTO 148 test samples were diluted serially in Iscove's, 1% BSA medium. Fifty microliters of target cells (˜10,000) and 100 microliters of antibody were added to a round bottom 96 well plate. Fifty microliters of effector cells (˜500,000 cells) were added to the mixture, and the plate was centrifuged at 1000 rpm for 5 min, RT. The effector cell to target cell ratio (E:T) was 50:1. To measure background fluorescence, wells were incubated with a mix of effector cells and target cells in medium, with no antibody. To establish maximal fluorescence, 10 microliters of lysis solution (from Delfia EuTDA Cytotoxicity kit) was added to background wells. For the ADCC assay, cells were incubated at 37° C., 5% CO₂, for approximately 2 h. Twenty microliters of supernatant was transferred to a 96 well flat bottom plate. Two hundred microliters of Europium solution (Delfia EuTDA Cytotoxcity Kit) was added and the plate was put on a plate shaker for 10 min, RT. Fluorescence was measured in the time-resolved fluorometer, EnVision Instrument (Perkin-Elmer Life Sciences). The percentage of specific lysis in each sample was calculated according to the following formula: % Specific release=([experimental release—spontaneous release]÷[maximum release−spontaneous release])×100.

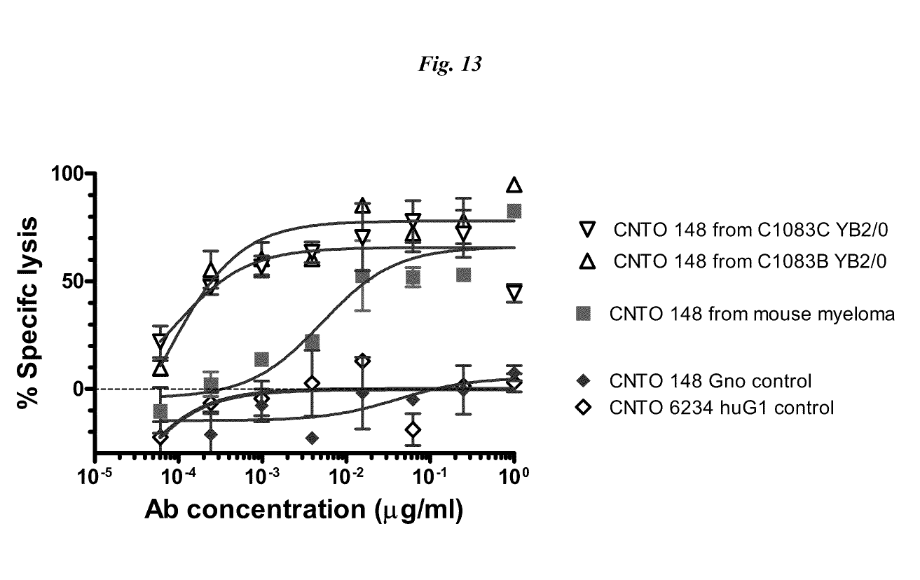

The results of the ADCC assays showed that the C1083B-derived CNTO 148 was approximately 70-fold more potent than the reference material, CNTO 148 from mouse myeloma cells (FIG. 13). The C1083C-derived CNTO 148 showed essentially the same potency as the C1083B-derived CNTO 148, consistent with the bioanalytical data that showed they had very similar levels of fucose. As a result of the unexpected similarity in fucose levels, these Ab lots did not offer a means to test whether extra low levels of fucose (10-20%) translated into no further enhancement of ADCC activity compared to having moderate levels of fucose (40-50%), as has been observed with CNTO 860 in vitro (and 2C11 in vivo). Nevertheless, these results provide another example of an Ab expressed in either C1083B or C1083C showing markedly enhanced ADCC activity relative to the same Ab expressed in an alternate host cell.

### Example 7

### In Vivo Agonist Activity of an Anti-CD3 AB Expressed in HEK 293E Cells, C1083A Cells, and C1083C Cells

Based on previous reports showing that T cell activation by anti-CD3 monoclonal Abs is dependent on the capacity of those Abs to bind Fcγ receptors (FcγRs), a simple model system was used to test whether mice would show differing degrees of an Fc-dependent response to a human IgG1 Ab with differing levels of fucose in its Fc glycan. Recombinant hamster anti-mouse CD3 ε-chain Ab, 145-2C11 (2C11), was used for these studies. A plasmid encoding a single-chain Fv version 2C11 was kindly provided by Dr. Jeffrey Bluestone (University of California, San Francisco). The heavy and light chain variable (V) region coding sequences in this plasmid were previously PCR-amplified and the amplified DNA fragments cloned first into genomic heavy and light chain V region vectors, and then into genomic constant region expression vectors for mouse IgG2a and kappa chains, respectively.

To prepare human IgG1 variants of 2C11, DNA encoding the heavy chain variable region was amplified from one of the previously-prepared plasmids, p2213, and cloned into two different expression vectors containing human G1 constant region coding sequence. This resulted in the generation of expression plasmids p2648, in which the Ab gene transcription was driven by a CMV promoter, and p2694, in which transcription was driven by a mouse immunoglobulin promoter. The 2C11 light chain variable region was amplified from plasmid p2208, and cloned into expression vectors containing human kappa constant regions, driven by either a CMV promoter or an immunoglobulin promoter. This resulted in the generation of expression plasmids p2623, in which the Ab gene transcription was driven by the CMV promoter, and p2669, in which transcription was driven by the immunoglobulin promoter. The CMV promoter-containing plasmids were expressed transiently in HEK 293E cells. Approximately 3.5×10⁸ cells were grown in a 10 tier cell stack (Corning) in growth media (DMEM with 10% FBS), overnight at 37° C. in 5% CO₂. A transfection cocktail prepared by mixing 1.4 ml of Lipofectamine 2000 with 300 ug each of plasmids p2648 or p2622 and p2623, in 40 ml of Optimem (Invitrogen, Inc.) was added to the cell stack, and incubated overnight at 37° C. The next day, media with transfection cocktail was replaced with 1 liter of 293 SFMII (Invitrogen, Inc.)+4 mM sodium butyrate, and the cells incubated for 4 days at 37° C. Supernatants containing expressed antibody was harvested, cleared by centrifugation and 0.8 micron filtration. Expressed antibody was purified by standard protein A affinity chromatography.

The immunoglobulin promoter-containing plasmids were introduced into C1083A and C1083C YB2/0 cells via stable transfections. Approximately 2×10⁷ YB2/0 cells were transfected by electroporation with 10 μg each of plasmids p2694 and p2669, and plated in 96-well cell culture dishes in growth media containing alpha MEM supplemented with 10% FBS, NEAA, L-glutamine, and sodium pyruvate. Cells were selected for stable integration of plasmids with mycophenolic acid. Antibody-secreting, mycophenolic acid-resistant clones were screened by anti-human IgG ELISA. High-expressing, stable clones were scaled up in culture medium containing 5% FBS. Expressed antibody was purified by standard protein A affinity chromatography.

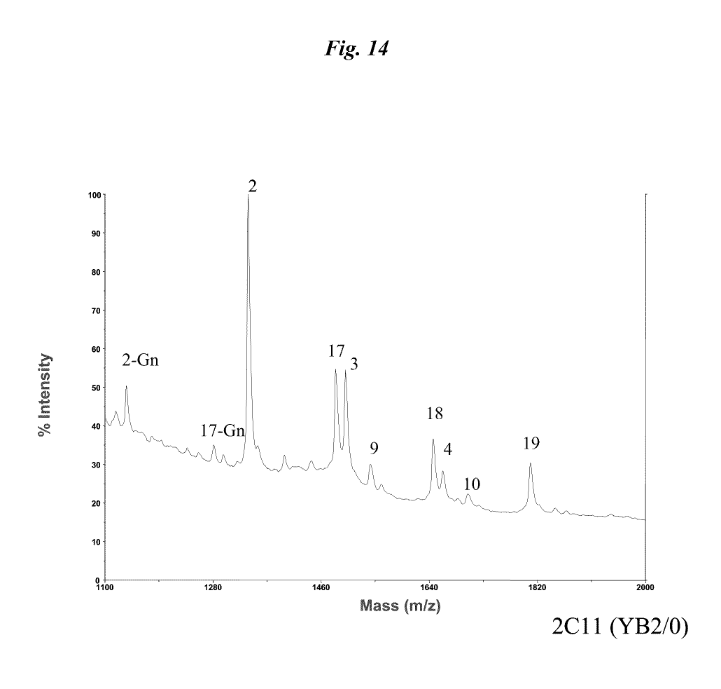

The prepared 2C11 huG1 Ab that had been expressed in C1083A YB2/0 cells was subjected to MALDO-TOF-MS as described in Examples 5 and 6 above (FIG. 14). This analysis demonstrated that the cell line, although cultured in the presence of serum, continued to produce glycosylated product Ab in which the dominant species is non-fucosylated (structure 2 as in FIG. 2). The 2C11 preparation was enzymatically deglycosylated in order to prepare a control Ab that lacked FcγR-binding capability. The deglycosylation was done by treating the Ab with 1000 Units of PNGase F at 37° C. for 24 h (˜10 mg Ab in 1.0 mL of buffer). Another aliquot of enzyme was added and the incubation was continued for an additional 24 h. The deglycosylated IgG samples were purified using a HiTrap Protein A column and formulated into phosphate-buffered saline, pH 7.0. The resulting glycoform, termed 2C11 Gno, was shown by MALDI-TOF-MS to have been thoroughly deglycosylated (not shown).

Concentrations of each Ab sample were determined by measuring OD₂₈₀ by spectrophotometry as well as staining of an SDS-polyacrylamide gel. LAL assays were performed on all test Abs to determine contaminating endotoxin levels. MALDI-TOF-MS and HPLC analyses performed as described above showed that the Fc glycan in the HEK 293E-derived Ab (2C11 huG1, HEK), the C1083A-derived Ab (2C11 huG1, C1083A), and the C1083C-derived Ab (2C11 huG1, C1083C) was approximately 95%, 40%, and 15% fucosylated, respectively. Quantitative binding analyses to CD3 on freshly-isolated mouse splenocytes revealed no detectable differences in antigen affinity for the three different Ab preps (data not shown).

To evaluate how the three Abs compared to each other with respect to their in vivo T cell activation properties, normal female Balb/c mice (Charles River Laboratories) were administered single intraperitoneal injections of varying amounts of test Ab. Approximately 24 hrs after test Ab injection, all mice were euthanized by CO₂ asphyxiation, terminal blood samples were collected via cardiac puncture, and spleens were harvested and placed into tubes containing cold harvest medium (RPMI 1640, 5% heat-inactivated fetal bovine serum, 1% L-glutamine). Single cell suspensions of the splenocytes were prepared by gently pressing the spleens through a 100 μm nylon mesh sieve and washing once with RPMI-1640 medium. The single cell suspension was then depleted of anucleated red blood cells using NH₄Cl hypotonic lyse solution, as per the manufacturer's instructions (Pharmingen). Splenocytes were washed twice and resuspended in PBS, 0.5% BSA with 0.2% sodium azide. Splenocytes were immunostained using CD4 PE⁺/CD25 APC⁺/CD8 and 7-AAD viability dye and analyzed by flow cytometry. All staining was done in the presence of the anti-CD 16/CD32 mAb, 2.4G2, to block staining mediated by Fc receptor binding.

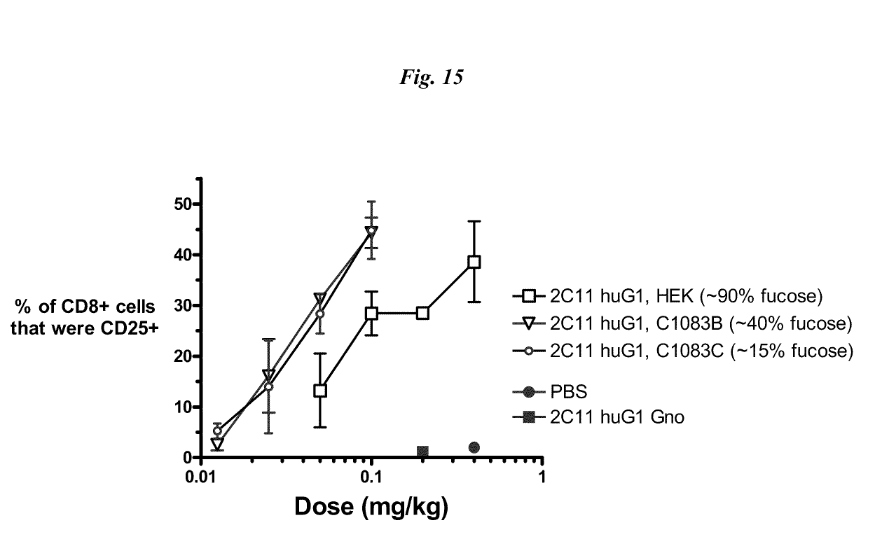

The results revealed greater T cell activation in mice dosed with the moderate-fucose variant compared to the high-fucose variant, with the high-fucose variant needing to be dosed with approximately 4 times more Ab to achieve the same degree of T cell activation (FIG. 15). However, the low-fucose variant was no more active than the moderate-fucose variant, suggesting that the complete absence of fucose is not necessary to achieve maximally enhanced Fc function of low-fucose variants in mice. Given that one of the human low-affinity FcγRs, FcγRIIIA, is sensitive to Fc fucose levels, these findings suggest that mice may more closely mimic Fc-dependent responses by human cells than previously thought.

All publications and patents mentioned herein are incorporated herein by reference for the purpose of describing and disclosing, for example, the constructs and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is to be claimed:

1. An isolated cell line derived from a rat myeloma cell line YB2/0 (ATCC 1662) useful for the production of an antibody, said cell line producing glycosylated polypeptides characterized as having substantially reduced content of fucose as compared to polypeptides produced using YB2/0 (ATCC 1662).

2. The cell line according to claim 1, wherein the cell line is developed from rat hybridoma cell line YB2/0 (C1083A) by adapting the cell line to grow in Animal-Protein-Free medium, CD-Hybridoma (CD-Hyb) and is designated C1083B.

3. The cell line according to claim 1, wherein the cell line is a subclone of C1083B selected based on at least one of high transfection efficiency, short mean doubling time and ability to reach high cell density in CD-Hyb and wherein the cell line is designated C1083E.

4. The cell line according to claim 1, wherein fut8 mRNA levels are lower than the levels of the wild-type YB2/0 cell line.

5. The cell line according to claim 1, wherein the cell line is selected for resistance to lectin.

6. The cell line according to claim 2, wherein the glycosylated peptides of the cell line have substantially reduced content of fucose as compared to peptides produced by wild-type myeloma cell lines and CHO cell lines.

7. An antibody produced by a transfected host cell line of any of claims 1-6, wherein the molecule is characterized as having predominantly non-fucosylated N-linked oligosaccharide groups.

8. The antibody of claim 7, wherein the antibody has increased ADCC activity compared to an anti-tissue factor antibody produced in a wild-type YB2/0 cell line.

9. A biopharmaceutical composition comprising the antibody of claim 7 in combination with a pharmaceutically acceptable carrier.

10. A method of producing an antibody, comprising:
transfecting a polynucleotide sequence encoding for the antibody into the cell line of claim 1; and
expressing the antibody in detectable or recoverable amounts.

11. A method of producing an antibody according to claim 10, wherein the antibody encoded by the polynucleotide sequence is a human antibody.

12. A method of producing an antibody according to claim 10, wherein the antibody encoded by the polynucleotide sequence is a humanized antibody.

13. A method of producing an antibody according to claim 11 or 12, wherein the antibody encoded by the polynucleotide sequence binds to a region of a human polypeptide which may be attached to the surface of a cell.

14. An antibody produced by the method of claim 10, wherein the recovered antibody encoded by the polynucleotide sequence is characterized as having predominantly non-fucosylated N-linked oligosaccharide groups.

15. An antibody produced by the method of claim 10, comprising a light chain amino acid sequence of SEQ ID NO:9 and a heavy chain amino acid sequence of SEQ ID NO:8.

16. An antibody produced by the method of claim 10, comprising a light chain variable region amino acid sequence of SEQ ID NO: 11 and a heavy chain variable region amino acid sequence of SEQ ID NO:10.

17. A method of treating a disease or condition, comprising administering or contacting a subject, cell, or tissue with the antibody of any of claims 14-16.

18. The method according to claim 17, wherein the disease or condition is a neoplastic disease or an immune-mediated disorder wherein the destruction of a cell displaying a polypeptide to which the antibody is capable of binding is desired.

19. The method according to claim 18, wherein the polypeptide to which the antibody is capable of binding is human tissue factor or human TNFalpha.

20. The method according to claim 18, wherein the disease or condition is characterized by abnormal angiogenesis selected from the group consisting of rheumatoid arthritis, macular degeneration, psoriasis, and diabetic retinopathy.

21. The method according to claim 19, wherein the disease or condition is characterized by release of said polypeptide from said cell.

22. Any invention disclosed herein.
